SEQUENCE LISTING (142 sequences)

SEQ ID NO: 1 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagaagattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 2 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagaggattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 3 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 4 - DNA, 785 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagatttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 5 - DNA, 784 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 6 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgaaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 7 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgagaagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 8 - DNA, 775 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 9 - DNA, 786 bp, Artificial Sequence
  Synthetic
atgatcgaaacatacaaccaaacttctccctgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 10 - DNA, 782 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 11 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagatttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 12 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgaggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 13 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgtggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 14 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcatatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 15 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcgtaagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 16 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagagaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 17 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtggggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 18 - DNA, 786 bp, Artificial Sequence
  Synthetic
atgatcgaaacatacaaccaaacttctccccgatctgtggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 19 - DNA, 783 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 20 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagggggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 21 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtaggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 22 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtggcctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 23 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaacggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 24 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaagaatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 25 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaacatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 26 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaacccaggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 27 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccccggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 28 - DNA, 785 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaaagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 29 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctgttatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 30 - DNA, 783 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 31 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcctaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 32 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcaggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 33 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatccggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 34 - DNA, 785 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaactccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 35 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccctggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 36 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaataaccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 37 - DNA, 785 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacattccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 38 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcaggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 39 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggtaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 40 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaataatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 41 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgagaggagtatttgaattgcaaccaggtgcttcggtgtttgtcaatgtgactgatccaagccaagtgagccatggcactggcttcacgtcctttggcttactcaaactctga

SEQ ID NO: 42 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggagtagtatttgaattgcaaccaggtgcttcggtgtttgtcaatgtgactgatccaagccaagtgagccatggcactggcttcacgtcctttggcttactcaaactctga

SEQ ID NO: 43 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtgtttgaattgcaaccaggtgcttcggtgtttgtcaatgtgactgatccaagccaagtgagccatggcactggcttcacgtcctttggcttactcaaactctga

SEQ ID NO: 44 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatctgaattgcaaccaggtgcttcggtgtttgtcaatgtgactgatccaagccaagtgagccatggcactggcttcacgtcctttggcttactcaaactctga

SEQ ID NO: 45 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtccttcggtgtttgtcaatgtgactgatccaagccaagtgagccatggcactggcttcacgtcctttggcttactcaaactctga

SEQ ID NO: 46 - DNA, 784 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcagtgactgatccaagccaagtgagccatggcactggcttcacgtcctttggcttactcaaactctga

SEQ ID NO: 47 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcaagtcctttggcttactcaaactctga

SEQ ID NO: 48 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcatgtcctttggcttactcaaactctga

SEQ ID NO: 49 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctctggcttactcaaactctga

SEQ ID NO: 50 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggctcactcaaactctga

SEQ ID NO: 51 - DNA, 786 bp, Artificial Sequence
  Synthetic
atgatcaaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 52 - DNA, 786 bp, Artificial Sequence
  Synthetic
atgatcgaaacatacaaccaaacttctccccgatctgcggccactggactgcccatcagcatgaaaatttttatgtatttgcttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 53 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagataattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 54 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattggatcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 55 - DNA, 785 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 56 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagacccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 57 - DNA, 786 bp, Artificial Sequence
  Synthetic
atgatcgaaacatacaaccaaacttttccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 58 - DNA, 786 bp, Artificial Sequence
  Synthetic
atgatcgaaacatacaaccaaacttctccccaatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 59 - DNA, 785 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 60 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcagcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 61 - DNA, 785 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacagtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 62 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagcgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 63 - DNA, 785 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgagccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 64 - DNA, 785 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaaaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 65 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagcgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 66 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaagatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 67 - DNA, 785 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 68 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccattaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 69 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatccatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 70 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagctacgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 71 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagctttgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 72 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagttaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 73 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctgcatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 74 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccagtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 75 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagactcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 76 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagacaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 77 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgtaaccaggtgcttcggtgtttgtcaatgtgactgatccaagccaagtgagccatggcactggcttcacgtcctttggcttactcaaactctga

SEQ ID NO: 78 - DNA, 786 bp, Artificial Sequence
  Synthetic
atgattgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 79 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgctttggtgtttgtcaatgtgactgatccaagccaagtgagccatggcactggcttcacgtcctttggcttactcaaactctga

SEQ ID NO: 80 - DNA, 786 bp, Artificial Sequence
  Synthetic
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttgacttactcaaactctga

SEQ ID NO: 81 - DNA, 786 bp, Artificial Sequence
  Synthetic
atgatcgaaacatacaaccaaacttctccccgatctgcggccactggactgcccatcagcatgaaaatttttatgtatttacttactgtttttcttaacacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 82 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 83 - protein, 261 aa, Artificial Sequence
  Synthetic
MIETYNQTSPRSAATGLPISMKIFMYLLTVFLITQRIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 84 - protein, 261 aa, Artificial Sequence; identical to SEQ ID NO: 82
  Synthetic

SEQ ID NO: 85 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEKIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 86 - protein, 261 aa, Artificial Sequence; identical to SEQ ID NO: 85
  Synthetic

SEQ ID NO: 87 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENRFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 88 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIEAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 89 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIVAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 90 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAYVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 91 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVVSEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 92 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVIREASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 93 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISGASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 94 - protein, 261 aa, Artificial Sequence
  Synthetic
MIETYNQTSPRSVATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 95 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQGAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 96 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWPEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 97 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAENGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 98 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKEYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 99 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYNMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 100 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTQENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 101 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTPENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 102 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLCYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 103 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNQEASSQAPFIASLCLKSPGRFERILLRAANTHSSAKPCGQQSIHLGGVFELQPGASVFVNVTDPSQVSHGTGFTSFGLLKL

SEQ ID NO: 104 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNPEASSQAPFIASLCLKSPGRFERILLRAANTHSSAKPCGQQSIHLGGVFELQPGASVFVNVTDPSQVSHGTGFTSFGLLKL

SEQ ID NO: 105 - protein, 261 aa, Artificial Sequence; identical to SEQ ID NO: 82
  Synthetic

SEQ ID NO: 106 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANNHSSAKPCGQQSIHLGGVFELQPGASVFVNVTDPSQVSHGTGFTSFGLLKL

SEQ ID NO: 107 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCRQQSIHLGGVFELQPGASVFVNVTDPSQVSHGTGFTSFGLLKL

SEQ ID NO: 108 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLRGVFELQPGASVFVNVTDPSQVSHGTGFTSFGLLKL

SEQ ID NO: 109 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGVVFELQPGASVFVNVTDPSQVSHGTGFTSFGLLKL

SEQ ID NO: 110 - protein, 261 aa, Artificial Sequence; identical to SEQ ID NO: 82
  Synthetic

SEQ ID NO: 111 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVSELQPGASVFVNVTDPSQVSHGTGFTSFGLLKL

SEQ ID NO: 112 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGPSVFVNVTDPSQVSHGTGFTSFGLLKL

SEQ ID NO: 113 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFKSFGLLKL

SEQ ID NO: 114 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFMSFGLLKL

SEQ ID NO: 115 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSSGLLKL

SEQ ID NO: 116 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGSLKL

SEQ ID NO: 117 - protein, 261 aa, Artificial Sequence
  Synthetic
MIKTYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 118 - protein, 261 aa, Artificial Sequence; identical to SEQ ID NO: 82
  Synthetic

SEQ ID NO: 119 - protein, 261 aa, Artificial Sequence
  Synthetic
MIETYNQTSPRSAATGLPISMKIFMYLLTVFLITQIIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 120 - protein, 261 aa, Artificial Sequence; identical to SEQ ID NO: 82
  Synthetic

SEQ ID NO: 121 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERPLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 122 - protein, 261 aa, Artificial Sequence
  Synthetic
MIETYNQTFPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 123 - protein, 261 aa, Artificial Sequence
  Synthetic
MIETYNQTSPQSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 124 - protein, 261 aa, Artificial Sequence; identical to SEQ ID NO: 82
  Synthetic

SEQ ID NO: 125 - protein, 261 aa, Artificial Sequence; identical to SEQ ID NO: 82
  Synthetic

SEQ ID NO: 126 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQRAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 127 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKRYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 128 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTIKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 129 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIHAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 130 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREATSQAPFIASLCLKSPGRFERILLRAANTHSSAKPCGQQSIHLGGVFELQPGASVFVNVTDPSQVSHGTGFTSFGLLKL

SEQ ID NO: 131 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREALSQAPFIASLCLKSPGRFERILLRAANTHSSAKPCGQQSIHLGGVFELQPGASVFVNVTDPSQVSHGTGFTSFGLLKL

SEQ ID NO: 132 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAAFIASLCLKSPGRFERILLRAANTHSSAKPCGQQSIHLGGVFELQPGASVFVNVTDPSQVSHGTGFTSFGLLKL

SEQ ID NO: 133 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPSRFERILLRAANTHSSAKPCGQQSIHLGGVFELQPGASVFVNVTDPSQVSHGTGFTSFGLLKL

SEQ ID NO: 134 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRLERILLRAANTHSSAKPCGQQSIHLGGVFELQPGASVFVNVTDPSQVSHGTGFTSFGLLKL

SEQ ID NO: 135 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFETILLRAANTHSSAKPCGQQSIHLGGVFELQPGASVFVNVTDPSQVSHGTGFTSFGLLKL

SEQ ID NO: 136 - protein, 261 aa, Artificial Sequence; identical to SEQ ID NO: 82
  Synthetic

SEQ ID NO: 137 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGALVFVNVTDPSQVSHGTGFTSFGLLKL

SEQ ID NO: 138 - protein, 261 aa, Artificial Sequence
  Synthetic
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFDLLKL

SEQ ID NO: 139 - protein, 261 aa, Artificial Sequence
  Synthetic
MIETYNQTSPRSAATGLPISMKIFMYLLTVFLNTQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 140 - protein, 219 aa, Artificial Sequence
  Synthetic
VGDGSSHHHHHHSSGGGRGSHHHHHHGGACGCAAAPDIKDLLSRLEELEGLVSSLREQGGGSGGGSGGGSMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 141 - protein, 453 aa, Artificial Sequence
  Synthetic
EVQLQQFGAELVKPGASVKISCKASGYTFTDYNMDWVKQSHGKSLEWIGDINPNYGSTSYNQKFKGKATLTVDKSSSTAYMELRSLTSEDTAVYYCARDWTGAMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 142 - protein, 220 aa, Artificial Sequence
  Synthetic
DIVMTQSPSSLSVSAGEKVTMNCKSSQSLLNSGNQRNYLAWYQQKPGQPPKLLIHGASTRESGVPDRFTGSGSGTDFTLTISSVQAEDLAVYYCQNDHRYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2022/0331362 A1
(43) Pub. Date: Oct. 20, 2022

(54) METHODS OF B CELL EXPANSION FOR USE IN CELL THERAPY

(71) Applicant: Walking Fish Therapeutics, South San Francisco, CA (US)

(72) Inventor: Thomas Brennan, Cupertino, CA (US)

(21) Appl. No.: 17/724,423

(22) Filed: Apr. 19, 2022

Related U.S. Application Data
(63) Continuation of application No. PCT/US22/25252, filed on Apr. 18, 2022.
(60) Provisional application No. 63/176,463, filed on Apr. 19, 2021.

(51) Int. Cl. A61K 35/17; C12N 5/0781; C12N 15/62; C12N 15/86; C07K 14/705; C07K 16/28; C07K 14/715; C07K 14/47

(52) U.S. Cl. CPC A61K 35/17; C12N 5/0635; C12N 15/625; C12N 15/86; C07K 14/70575; C07K 16/2875; C07K 14/70564; C07K 14/715; C07K 14/70532; C07K 14/70525; C07K 14/70528; C07K 14/70578; C07K 14/705; C07K 14/47; C07K 14/70517; C07K 14/70521; C07K 14/70503; C12N 2501/2304; C12N 2501/2321; C12N 2502/99; C12N 2710/10042; C07K 2317/56; C07K 2319/00; C07K 2317/622

(57) ABSTRACT

The invention disclosed herein relates to improved methods for expanding cell populations, particularly B cell populations. The invention further relates to improved B-cell expansion media, compositions comprising expanded B cells and methods of using such expanded B cells. The invention further relates to methods of treating diseases or disorders wherein a population of B cells is obtained and cultured, and wherein said B cells are engineered to express a payload and/or a chimeric receptor, and wherein said B cells are administered to a subject.

### CROSS REFERENCE TO RELATED APPLICATIONS

This application is a continuation of International Patent Application No. PCT/US22/25252, filed on Apr. 18, 2022, and claims priority to U.S. Provisional Patent Application No. 63/176,463, filed on Apr. 19, 2021, the contents of each of which are incorporated by reference herein in their entireties.

### SEQUENCE LISTING

The application contains a Sequence Listing that has been filed electronically in the form of a text file, created Apr. 18, 2022, and named “109036-0059_Sequence_Listing.TXT” (240,276 bytes), the contents of which are incorporated by reference herein in their entirety.

### BACKGROUND OF THE INVENTION

B cells are immune cells responsible for a variety of functions including helping the body resist infection and disease. They are capable of secreting antibodies in response to a recognized antigen, presenting antigens, and also secreting cytokines. In cancer, B cells have been found in tertiary lymphoid structures (“TLSs”) surrounding certain tumors. TLSs comprise aggregates of immune cells (including both T and B cells). Their presence in tumors is associated with better patient outcomes. See, e.g., Helmink, B. A., et al., Nature, 2020, 577(7791), 549-555; Petitprez F et al., Nature, 2020, 577(7791), 556-560.

Intratumoral injection of LPS-activated spleen cells, which include B cells, in combination with checkpoint inhibitors has been shown to produce anti-tumor responses. Soldevilla et al., Oncoimmunology, 2018, 7:8, e1450711. Further, given the natural ability of B cells to present antigens and secrete proteins, there is great potential as a cellular therapy for targeting certain diseased cell types and secreting therapeutic payloads.

Scaling the manufacturing of engineered B cells for cell therapy, however, is a challenging process. For several decades, activation and proliferation of B cells in vitro have been achieved through CD40-Ligand (“CD4OL”)-expressing feeder cell layer systems. Banchereau, J. et al. (1991) Science 251: 70-72; Schultze, J. L. et al. (1997) J. Clin. Invest. 100: 2757-2765; Liebig, T. M. et al. (2009) J. Vis. Exp. 32: 1373. These systems share a common drawback, which is their dependence on CD40L-expressing feeder cell layers. Use of feeder cells impedes standardization of the activation and proliferation process and introduces a variable into the protocol.

Expansion of B cells has been described using CD40L, with mixed results. See e.g., Wennhold et al., 2019, Transfus Med Hemother, 46:36-46. Some methods result in low expansion yields, producing quantities of B cells in insufficient quantities for cellular engineering and for using B cells as a therapeutic for administration to patients. Accordingly, there exists a need for improved B cell expansion techniques, growth media, and the like.

### SUMMARY OF THE INVENTION

The invention generally provides improved methods for expanding cell populations, particularly B cell populations. The invention further relates to improved cell media, compositions thereof, and methods of using such expanded B cells. This novel method incorporates the use of a novel CD40L fusion protein, a cross-linking antibody, and IL-4 and/or IL-21. Such methods are shown here to be crucial for effective activation and proliferation of engineered B cells, resulting in a 200-fold increase in the desired levels of functionally expanded B cells.

The methods provided herein for expanding B cells are superior to conventional methods in that conventional expansion methods to date result in undesirably low cell expansion, and thus reduced yield of engineered B cells. Proper cell function and yields are critical in cell therapy, for allogeneic treatments, and particularly in the autologous treatment setting where there is frequently only a single opportunity to harvest the patient's cells, culture, expand, engineer and administer an efficacious dose of such cells.

In various embodiments, the invention relates to a method of treating a disease or disorder in a subject in need thereof, comprising obtaining a population of B cells from a source, culturing said B cells in a culture medium comprising a CD40L fusion protein and a CD40L cross-linking agent, engineering said B cells to express either a payload, a chimeric receptor, or both; and administering said B cells to said subject. In various embodiments, the source is a mammal. In various embodiments, said source is a biological sample comprising peripheral mononuclear blood cells.

In various embodiments, the CD40L fusion protein comprises an amino acid sequence at least 85% identical to SEQ ID NO. 3. In various embodiments, the CD40L comprises an amino acid sequence at least 95% identical to SEQ ID NO. 3. In various embodiments, the CD40L fusion protein comprises an amino acid sequence of SEQ ID NO. 3 fusion protein. In various embodiments, the CD40L crosslinking agent is an antibody. In various embodiments, the antibody comprises a light chain variable region comprising the amino acid sequence at least 95% identical to SEQ ID NO. 5 and a heavy chain variable region comprising the amino acid sequence at least 95% identical to SEQ ID NO. 7. In various embodiments, the antibody comprises a light chain variable region comprising the amino acid sequence of SEQ ID NO. 5 and a heavy chain variable region comprising the amino acid sequence of SEQ ID NO. 7.

In various embodiments, the B cell is engineered prior to culturing said B cells in a medium with CD40L fusion protein and a CD40L crosslinking agent. In various embodiments, the B cell is engineered after culturing said B cells in a medium with CD40L fusion protein and a CD40L crosslinking agent. In various embodiments, the method involves further culturing said B cells in the presence of IL-4. In various embodiments, the method involves further culturing said B cells in the presence of IL-21.

In various embodiments, the cultured B cells express at least one of the following markers: CD62L, CCR7, CD80, CD86, CD54, ICAM, CD58, or CD27. In various embodiments, the disease or disorder is selected from the group consisting of at least one of cancer, heart disease, inflammatory disease, muscle-wasting disease, or neurological disease. In various embodiments the cancer is at least one of breast cancer, colon cancer, rectal cancer, esophageal cancer; lung cancer, pancreatic cancer, stomach cancer, liver cancer, hepatocellular carcinoma, stromal tumors such as GIST, glioblastoma, and glioma. In various embodiments, at least about 3×10⁷ B cells are administered to said subject. In various embodiments, the population of B cells are cultured for at least 14 days.

In various embodiments, the present invention relates to a method of treating a disease or disorder in a subject in need thereof comprising obtaining a population of B cells from a source; culturing said B cells in a culture medium comprising a CD40L fusion protein, wherein said CD40L fusion protein comprises an amino acid sequence at least 95% identical to the amino acid sequence of SEQ ID NO. 3, and a CD40L cross-linking antibody whose light chain variable region is at least 95% identical to the amino acid sequence of SEQ ID NO: 5 and whose heavy chain variable region is at least 95% identical to the amino acid sequence of SEQ ID NO: 7; and administering said B cells to said subject.

In various embodiments, the source is a mammal. In various embodiments, the CD40L fusion protein comprises an amino acid sequence of SEQ ID NO. 3. In various embodiments, the antibody comprises a light chain variable region comprising the amino acid sequence of SEQ ID NO. 5 and a heavy chain variable region comprising the amino acid sequence of SEQ ID NO. 7. In various embodiments, the invention further comprises engineering said B cells to express either a payload, a chimeric receptor or both.

In various embodiments, the B cell is engineered prior to culturing said B cells in a medium with CD40L and a CD40L crosslinking antibody. In various embodiments, the B cell is engineered after culturing said B cells in a medium with CD40L and a CD40L crosslinking antibody. In various embodiments, the invention further comprises culturing said B cells in the presence of IL-4. In various embodiments, the invention further comprises culturing said B cells in the presence of IL-21. In various embodiments, the cultured B cells express at least one of the following markers: CD62L, CCR7, CD80, CD86, CD54, ICAM, CD58, or CD27. In various embodiments, the disease or disorder is selected from the group consisting of at least one of cancer, heart disease, inflammatory disease, muscle wasting disease, or neurological disease. In various embodiments, the cancer is at least one of breast cancer, colon cancer, rectal cancer, esophageal cancer; lung cancer, pancreatic cancer, stomach cancer, liver cancer, hepatocellular carcinoma, stromal tumors such as GIST, glioblastoma, and glioma. In various embodiments, at least about 3×10⁷ B cells are administered to said subject. In various embodiments, the population of B cells are cultured for at least 14 days.

In various embodiments, the present invention relates to a method for manufacturing engineered B cells, said method comprising obtaining a population of B cells from a source, culturing said B cells in a culture medium comprising CD40L fusion protein and a CD40L cross-linking agent, and engineering said B cells to express either a payload, a chimeric receptor, or both. In various embodiments, the source is a mammal. In various embodiments, said source is a biological sample comprising peripheral mononuclear blood cells.

In various embodiments, the CD40L fusion protein comprises an amino acid sequence at least 85% identical to SEQ ID NO. 3. In various embodiments, the CD40L comprises an amino acid sequence at least 95% identical to SEQ ID NO. 3. In various embodiments, the CD40L fusion protein comprises an amino acid sequence of SEQ ID NO. 3 fusion protein. In various embodiments, the CD40L crosslinking agent is an antibody. In various embodiments, the antibody comprises a light chain variable region comprising the amino acid sequence at least 95% identical to SEQ ID NO. 5 and a heavy chain variable region comprising the amino acid sequence at least 95% identical to SEQ ID NO. 7. In various embodiments, the antibody comprises a light chain variable region comprising the amino acid sequence of SEQ ID NO. 5 and a heavy chain variable region comprising the amino acid sequence of SEQ ID NO. 7.

In various embodiments, the B cell is engineered prior to culturing said B cells in a medium with CD40L and a CD40L crosslinking agent. In various embodiments, the B cell is engineered after culturing said B cells in a medium with CD40L and a CD40L crosslinking agent. In various embodiments, the method involves further culturing said B cells in the presence of IL-4. In various embodiments, the method involves further culturing said B cells in the presence of IL-21. In various embodiments, the cultured B cells express at least one of the following markers: CD62L, CCR7, CD80, CD86, CD54, ICAM, CD58, or CD27. In various embodiments, the source is a mammal. In various embodiments, the source is a biological sample comprising peripheral mononuclear blood cells.

In various embodiments, the CD40L fusion protein comprises an amino acid sequence at least 85% identical to SEQ ID NO. 3. In various embodiments, the CD40L comprises an amino acid sequence at least 95% identical to SEQ ID NO. 3. In various embodiments, the CD40L fusion protein comprises an amino acid sequence of SEQ ID NO. 3 fusion protein. In various embodiments, the CD40L crosslinking agent is an antibody. In various embodiments, the antibody comprises a light chain variable region comprising the amino acid sequence at least 95% identical to SEQ ID NO. 5 and a heavy chain variable region comprising the amino acid sequence at least 95% identical to SEQ ID NO. 7. In various embodiments, the antibody comprises a light chain variable region comprising the amino acid sequence of SEQ ID NO. 5 and a heavy chain variable region comprising the amino acid sequence of SEQ ID NO. 7.

In various embodiments, the method further comprises culturing said B cells in the presence of IL-4. In various embodiments, the method further comprises culturing said B cells in the presence of IL-21. In various embodiments, the cultured B cells express at least one of the following markers: CD62L, CCR7, CD80, CD86, CD54, ICAM, CD58, or CD27. In various embodiments, at least about 3×10⁷ B cells are obtained. In various embodiments, the population of B cells are cultured for at least 14 days.

In various embodiments, a method of manufacturing engineered B cells is provided, said method comprising obtaining a population of B cells from a source; culturing said B cells in a culture medium comprising a CD40L, wherein said CD40L fusion protein comprises an amino acid sequence at least 95% identical to the amino acid sequence of SEQ ID NO. 3, and a CD40L cross-linking antibody whose light chain variable region is at least 95% identical to the amino acid sequence of SEQ ID NO: 5 and whose heavy chain variable region is at least 95% identical to the amino acid sequence of SEQ ID NO: 7; and administering said B cells to said subject. In various embodiments, the source is a mammal. In various embodiments, the CD40L fusion protein comprises an amino acid sequence of SEQ ID NO. 3. In various embodiments, the antibody comprises a light chain variable region comprising the amino acid sequence of SEQ ID NO. 5 and a light chain variable region comprising the amino acid sequence of SEQ ID NO. 7.

In various embodiments, the method further comprises engineering said B cells to express either a payload, a chimeric receptor, or both. In various embodiments, the B cell is engineered prior to culturing said B cells in a medium with CD40L and a CD40L crosslinking agent. In various embodiments, the B cell is engineered after culturing said B cells in a medium with CD40L and a CD40L crosslinking agent. In various embodiments, the method further comprises culturing said B cells in the presence of IL-4. In various embodiments, the method further comprises culturing said B cells in the presence of IL-21. In various embodiments, the cultured B cells express at least one of the following markers: CD62L, CCR7, CD80, CD86, CD54, ICAM, CD58, or CD27. In various embodiments, at least about 3×10⁷ B cells are obtained. In various embodiments, the population of B cells are cultured for at least 14 days.

In various embodiments, the invention relates to a B-cell expansion media comprising a CD40L fusion protein, wherein said CD40L fusion protein comprises an amino acid sequence at least 95% identical to the amino acid sequence of SEQ ID NO. 3, and a CD40L cross-linking antibody whose light chain variable region is at least 95% identical to the amino acid sequence of SEQ ID NO: 5 and whose heavy chain variable region is at least 95% identical to the amino acid sequence of SEQ ID NO: 7; and administering said B cells to said subject.

In various embodiments, the CD40L fusion protein comprises an amino acid sequence of SEQ ID NO. 3. In various embodiments, the antibody comprises a light chain variable region comprising the amino acid sequence of SEQ ID NO. 5 and a heavy chain variable region comprising the amino acid sequence of SEQ ID NO. 7. In various embodiments, the media further comprises IL-4. In various embodiments, the media further comprises IL-21.

### BRIEF DESCRIPTION OF THE FIGURES

FIG. 1 shows that human B cells can be grown and expanded in media with HELA-CD40 feeder cells, but do not expand in the presence of MEGA-CD40L (Enzo Life Sciences). Purified B cells were started at equivalent densities on day 0 at approximately 10,000 cells per ml and grown on HELA-CD40L feeder cells. On day 4, the cultures were split into two groups and one group continued to be grown in the same media with HELA-CD40L feeder cells, whereas the other group was grown in media supplemented with MEGA-CD40L (0.1 μg/ml) but in the absence of HELA feeder cells. On day 8, an additional 100 ng/ml of MEGA-CD40L was added to the growth media in the MEGA-CD40L condition. Arrows indicate the time at which MEGA-CD40L treatment occurred. This data shows there was significant proliferation of B-cells by day 11 when grown on HELA-CD40L feeder cells but no significant growth was observed on day 11 if grown with MEGA-CD40L.

FIG. 2 shows that human-cells can be grown and expanded in media comprising CD40L and a CD40L cross-linking agent at least to about 3×10⁷ cells after just two weeks, even in the absence of HELA-CD40L feeder cells. Two densities were maintained throughout the course of the study (150K and 1MM). The lower density culture was able to maintain a higher growth rate and gave a larger relative total mass. Even in just two weeks, cells were able to expand at least 200-fold.

FIG. 3 demonstrates that expanded engineered B cells can maintain transgene expression. FIG. 3A demonstrates that 67% of the expanded, engineered B cells exhibited GPC B Cell Receptor expression 72 hours following adenoviral vector transfection. FIG. 3B shows the percentage of GPC-BCR expressing cells in cells transfected with an empty vector (one that did not express GPC3). FIG. 3C shows cells which were not transduced with any vector at all.

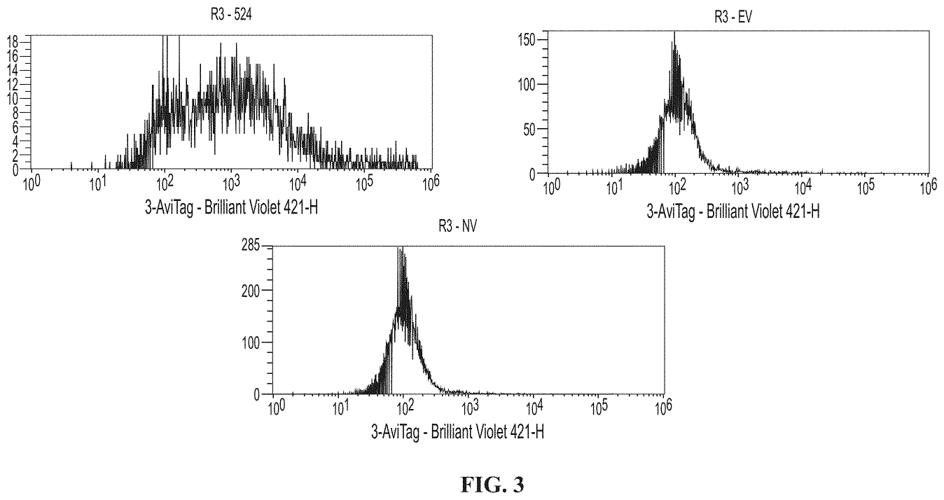

FIG. 4 shows B cell expansion and growth rate post Ad5RGD-GFP Transduction. Ad5RGD-GFP resulted in high efficiency of GFP expression in B cells. Expression was maintained in —60% of total B cells for at least 8-10 days. The GFP+B cells continued to proliferate for more than a week post transduction.

FIG. 5 shows successful expansion of Human B cells transduced with a murine GPC3 construct via RGD (601). FIG. 5A depicts the structure of the transduced anti-GPC3 scFV chimeric receptor, which comprises an anti-GPC3 scFv, a CD8 hinge domain, a CD28 transmembrane domain and a CD79a signaling domain. FIG. 5B demonstrates the relative percentage of B cells expressing the transduced GPC3 chimeric receptor.

FIG. 6 shows successful expansion of Human B cells transduced with a murine GPC3 construct via RGD (602). FIG. 6A depicts the structure of the transduced anti-GPC3 scFV chimeric receptor, which comprises an anti-GPC3 scFv, a CD8 hinge domain, a CD28 transmembrane domain and a CD79b signaling domain. FIG. 6B demonstrates the relative percentage of B cells expressing the transduced GPC3 chimeric receptor.

FIG. 7 shows expansion of human B cells transduced with an RGD-functionalized nonviral gene delivery vector expressing a chimeric receptor that targets GPC3. FIG. 7A depicts the structure of this chimeric receptor which comprises an anti-GPC3 scFv, a CD8 hinge domain, a CD28 transmembrane domain and a CD79b or CD79a signaling domain. FIG. 7B demonstrates the relative percentage of B cells expressing the transduced GPC3 chimeric receptor.

FIG. 8 shows successful expansion of Human B cells transduced with a murine GPC3 construct via RGD (463). FIG. 8A depicts the structure of the transduced anti-GPC3 scFV chimeric receptor, which comprises an anti-GPC3 scFv, a CD8 hinge domain, a CD28 transmembrane domain and a CD79b signaling domain. FIG. 8B demonstrates the relative percentage of B cells expressing the transduced GPC3 chimeric receptor.

FIG. 9 shows expansion of human B cells transduced with an RGD-functionalized nonviral gene delivery vector expressing a chimeric receptor that targets sarcoglycan (394). FIG. 9A depicts the structure of this chimeric receptor which comprises an anti-sarcoglycan scFv, a murine G2a Fc domain, a transmembrane domain, and a cytoplasmic tail. FIG. 9B demonstrates the percentage of cells demonstrated to express the chimeric receptor after transduction and expansion of B cells.

FIG. 10 shows in vivo homing of engineered and expanded human B cells to tumor-draining lymph nodes (“TDLN”) in mice harboring HPEG2 Tumors.

### DETAILED DESCRIPTION

It will be understood that descriptions herein are exemplary and explanatory only and are not restrictive of the invention as claimed. In this application, the use of the singular includes the plural unless specifically stated otherwise.

### I. Overview

It will be appreciated that the invention relates methods of producing, expanding and/or isolating populations of engineered B cells. For example, the instant methods can be utilized to produce for example a variety of engineered B cells as described in U.S. provisional patent application No. 63/073799 filed Sep. 2, 2020, and U.S. provisional patent application No. 63/003120 filed Mar. 31, 2020. These include, but are not limited to:

1) B cells that have been modified to home to a site/target of interest, using, e.g., a binding domain such as an scFv, antibody, ligand, receptor, or fragments thereof;

2) B cells that have been modified with a homing domain, further comprising an activation, and optionally a costimulatory domain, such that the B cells can home and activate upon interaction with a desired target;

3) B cells engineered to be capable of making a desired protein payload, such as an antibody, therapeutic protein, polypeptide, nucleic acid sequence (such as RNAi) or the like;

4) Engineered B cells comprising a homing/binding domain, an activating domain, an optional costimulatory domain, and further engineered to express a desire protein payload, such as an antibody, therapeutic protein, polypeptide, nucleic acid sequence (such as RNAi) or the like;

5) B cells that have been modified to express an integrin, a homing antibody, protein, or a receptor, such that the B cells are attracted to specific ligands, chemokines, or attractants at a specific site/target of interest (e.g., a homing tissue) and can thereby home to the site/target of interest, for example, to deliver a desired payload;

6) B cells that have been modified to express an immune inhibitory molecule, such that the inflammation and autoimmune activity of B cells localized to a site/target of interest is decreased, thereby leading to a positive therapeutic response;

7) B cells that have been treated with a compound or derivatives thereof, such that trafficking of the B cells is altered by expression of specific B cell integrins and/or homing receptors;

8) B cells that have been (i) treated with a Toll-like receptor (TLR) agonist, and/or (ii) engineered to express a constitutively active TLR, for potentiating B cells and/or producing potent effector B cells for increasing immune responses in a subject;

9) B cells that have been electroporated with an mRNA encoding specific antigens of interest fused to a targeting signal of a lysosomal protein, such that the B cells can simultaneously and efficiently present the specific antigens and/or antigen-derived epitopes of interest in both HLA class I and class II molecules.

10) B cells that have been electroporated with a self-amplifying RNA that encodes any items noted above in parts 1-9.

It will be understood that the various embodiments of engineered or modified B cells of the present application are not mutually exclusive and can be combined with each other in any way and without any restriction unless explicitly indicated, for achieving of facilitating any of the results and/or therapeutic responses contemplated herein.

More specifically, the present methods can be used as a manufacturing technique to produce improved expansion of B cells in the production of the following B cell embodiments. These are described in in U.S. provisional patent application No. 63/073799 filed Sep. 2, 2020, and U.S. provisional patent application No. 63/003120 filed Mar. 31, 2020, the contents of which are hereby incorporated by reference in their entirety. Certain methods for making constructs and engineered immune cells of the invention are described in PCT application PCT/US2015/14520, the contents of which are hereby incorporated by reference in their entirety. Additional methods of making the constructs and cells can be found in U.S. provisional patent application No. 62/244,036 the contents of which are further hereby incorporated by reference in their entirety.

The invention also relates to methods of treating a disease or disorder using engineered B cells produced by the methods described herein. Examples of diseases or disorders suitable for treatment include, but are not limited to cancer, heart disease, inflammatory disease, muscle wasting disease, neurological disease, and the like.

### II. Definitions

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. As utilized in accordance with the present disclosure, the following terms, unless otherwise indicated, shall be understood to have the following meanings:

In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The term “polynucleotide”, “nucleotide”, or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′, 3′-dideoxyribose, and internucleotide linkage modifications such as phosphorothioate, phosphorodithioate, phosphoroselenoate, phosphoro-diselenoate, phosphoro-anilothioate, phoshoraniladate and phosphoroamidate.

The term “oligonucleotide” refers to a polynucleotide comprising 200 or fewer nucleotides. Oligonucleotides can be single stranded or double stranded, e.g., for use in the construction of a mutant gene. Oligonucleotides can be sense or antisense oligonucleotides. An oligonucleotide can include a label, including a radiolabel, a fluorescent label, a hapten or an antigenic label, for detection assays. Oligonucleotides can be used, for example, as PCR primers, cloning primers or hybridization probes.

The term “control sequence” refers to a polynucleotide sequence that can affect the expression and processing of coding sequences to which it is ligated. The nature of such control sequences can depend upon the host organism. In particular embodiments, control sequences for prokaryotes can include a promoter, a ribosomal binding site, and a transcription termination sequence. For example, control sequences for eukaryotes can include promoters comprising one or a plurality of recognition sites for transcription factors, transcription enhancer sequences, and transcription termination sequence. “Control sequences” can include leader sequences (signal peptides) and/or fusion partner sequences.

As used herein, “operably linked” means that the components to which the term is applied are in a relationship that allows them to carry out their inherent functions under suitable conditions.

The term “vector” means any molecule or entity (e.g., nucleic acid, plasmid, bacteriophage or virus) used to transfer protein coding information into a host cell. The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct can include, but is not limited to, sequences that affect or control transcription, translation, and, if introns are present, affect RNA splicing of a coding region operably linked thereto.

The term “host cell” refers to a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell, or can be maintained transiently as an episomal element without being replicated, or can replicate independently as a plasmid. A cell is considered to have been “stably transformed” when the transforming DNA is replicated with the division of the cell.

The term “transfection” refers to the uptake of foreign or exogenous DNA by a cell. A number of transfection techniques are well known in the art and are disclosed herein. See, e.g., Graham et al., 1973, Virology, 1973, 52:456; Sambrook et al., Molecular Cloning: A Laboratory Manual, 2001, supra; Davis et al., Basic Methods in Molecular Biology, 1986, Elsevier; Chu et al., 1981, Gene, 13:197.

The term “transduction” refers to the process whereby foreign DNA is introduced into a cell via viral vector. See, e.g., Jones et al., Genetics: Principles and Analysis, 1998, Boston: Jones & Bartlett Publ.

The terms “polypeptide” or “protein” refer to a macromolecule having the amino acid sequence of a protein, including deletions from, additions to, and/or substitutions of one or more amino acids of the native sequence. The terms “polypeptide” and “protein” specifically encompass antigen-binding molecules, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. Useful polypeptide fragments include immunologically functional fragments of antigen-binding molecules.

The term “isolated” means (i) free of at least some other proteins with which it would normally be found, (ii) is essentially free of other proteins from the same source, e.g., from the same species, (iii) separated from at least about 50 percent of polynucleotides, lipids, carbohydrates, or other materials with which it is associated in nature, (iv) operably associated (by covalent or noncovalent interaction) with a polypeptide with which it is not associated in nature, or (v) does not occur in nature.

A “variant” of a polypeptide (e.g., an antigen-binding molecule) comprises an amino acid sequence wherein one or more amino acid residues are inserted into, deleted from and/or substituted into the amino acid sequence relative to another polypeptide sequence. Variants include fusion proteins.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”).

To calculate percent identity, the sequences being compared are typically aligned in a way that gives the largest match between the sequences. One example of a computer program that can be used to determine percent identity is the GCG program package, which includes GAP (Devereux et al., Nucl. Acid Res., 1984, 12, 387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). In certain embodiments, a standard comparison matrix (see, e.g., Dayhoff et al., 1978, Atlas of Protein Sequence and Structure, 5:345-352 for the PAM 250 comparison matrix; Henikoff et al., 1992, Proc. Natl. Acad. Sci. U.S.A., 89, 10915-10919 for the BLO-SUM 62 comparison matrix) is also used by the algorithm.

As used herein, the twenty conventional (e.g., naturally occurring) amino acids and their abbreviations follow conventional usage. See, e.g., Immunology A Synthesis (2nd Edition, Golub and Green, Eds., Sinauer Assoc., Sunderland, Mass. (1991)), which is incorporated herein by reference for any purpose. Stereoisomers (e.g., D-amino acids) of the twenty conventional amino acids, unnatural amino acids such as alpha-, alpha-disubstituted amino acids, N-alkyl amino acids, lactic acid, and other unconventional amino acids can also be suitable components for polypeptides of the present invention. Examples of unconventional amino acids include: 4-hydroxyproline, .gamma.-carboxy-glutamate, epsilon-N,N,N-trimethyllysine, e-N-acetyllysine, 0-phosphoserine, N-acetylserine, N-formylmethionine, 3-methylhistidine, 5-hydroxylysine, .sigma.-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Conservative amino acid substitutions can encompass non-naturally occurring amino acid residues, which are typically incorporated by chemical peptide synthesis rather than by synthesis in biological systems. These include peptidomimetics and other reversed or inverted forms of amino acid moieties. Naturally occurring residues can be divided into classes based on common side chain properties:
a) hydrophobic: norleucine, Met, Ala, Val, Leu, Ile;
b) neutral hydrophilic: Cys, Ser, Thr, Asn, Gln;
c) acidic: Asp, Glu;
d) basic: His, Lys, Arg;
e) residues that influence chain orientation: Gly, Pro; and
f) aromatic: Trp, Tyr, Phe.

For example, non-conservative substitutions can involve the exchange of a member of one of these classes for a member from another class.

In making changes to the antigen-binding molecule, the costimulatory or activating domains of the engineered T cell, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). See, e.g., Kyte et al., 1982, J. Mol. Biol., 157, 105-131. It is known that certain amino acids can be substituted for other amino acids having a similar hydropathic index or score and still retain a similar biological activity. It is also understood in the art that the substitution of like amino acids can be made effectively on the basis of hydrophilicity, particularly where the biologically functional protein or peptide thereby created is intended for use in immunological embodiments, as in the present case. Exemplary amino acid substitutions are set forth in Table 1.

TABLE 1

| Original | | |
|---|---|---|
| Residues | Exemplary Substitutions | Preferred Substitutions |
| Ala | Val, Leu, Ile | Val |
| Arg | Lys, Gin, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Va, Met, Ala, Phe | Ile |
| Lys | Arg, 1, 4 Diamino-butyric | Arg |
| | Acid, Gin, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen-binding molecule can have a greater circulating half-life than an antigen-binding molecule that is not chemically modified. In some embodiments, a derivative antigen-binding molecule is covalently modified to include one or more water-soluble polymer attachments, including, but not limited to, polyethylene glycol, polyoxyethylene glycol, or polypropylene glycol.

Peptide analogs are commonly used in the pharmaceutical industry as non-peptide drugs with properties analogous to those of the template peptide. These types of non-peptide compound are termed “peptide mimetics” or “peptidomimetics.” Fauchere, J. L., 1986, Adv. Drug Res., 1986, 15, 29; Veber, D. F. & Freidinger, R. M., 1985, Trends in Neuroscience, 8, 392-396; and Evans, B. E., et al., 1987, J. Med. Chem., 30, 1229-1239, which are incorporated herein by reference for any purpose.

The term “therapeutically effective amount” refers to the amount of immune cells or other therapeutic agent determined to produce a therapeutic response in a mammal. Such therapeutically effective amounts are readily ascertained by one of ordinary skill in the art.

The terms “patient” and “subject” are used interchangeably and include human and non-human animal subjects as well as those with formally diagnosed disorders, those without formally recognized disorders, those receiving medical attention, those at risk of developing the disorders, etc.

The term “treat” and “treatment” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. The term “prevent” does not require the 100% elimination of the possibility of an event. Rather, it denotes that the likelihood of the occurrence of the event has been reduced in the presence of the compound or method.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% higher compared to a reference quantity, level, value, number, frequency, percentage, dimension, size, amount, weight or length. In one embodiment, the terms “essentially the same” or “substantially the same” refer to a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell population or culture media, refer to a composition that is free of a specified substance, such as, 95% free, 96% free, 97% free, 98% free, 99% free of the specified substance, or is undetectable as measured by conventional means. Similar meaning can be applied to the term “absence of,” where referring to the absence of a particular substance or component of a composition.

As used herein, the term “appreciable” refers to a range of quantity, level, value, number, frequency, percentage, dimension, size, amount, weight or length or an event that is readily detectable by one or more standard methods. The terms “not-appreciable” and “not appreciable” and equivalents refer to a range of quantity, level, value, number, frequency, percentage, dimension, size, amount, weight or length or an event that is not readily detectable or undetectable by standard methods. In one embodiment, an event is not appreciable if it occurs less than 5%, 4%, 3%, 2%, 1%, 0.1%, 0.001%, or less of the time.

Throughout this specification, unless the context requires otherwise, the words “comprise,” “comprises” and “comprising” will be understood to imply the inclusion of stated step or element or group of steps or elements but not the exclusion of any other step or element or group of steps or elements. In particular embodiments, the terms “include,” “has,” “contains,” and “comprise” are used synonymously.

As used herein, “consisting of” is meant including, and limited to, whatever follows the phrase “consisting of”. Thus, the phrase “consisting of” indicates that the listed elements are required or mandatory, and that no other elements may be present.

By “consisting essentially or” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” or “approximately” refers to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5% or 1%, or any intervening ranges thereof.

As used herein, the term “introducing” refers to a process that comprises contacting a cell with a polynucleotide, polypeptide, or small molecule. An introducing step may also comprise microinjection of polynucleotides or polypeptides into the cell, use of liposomes to deliver polynucleotides or polypeptides into the cell, or fusion of polynucleotides or polypeptides to cell permeable moieties to introduce them into a cell.

### III. Methods of Expanding Populations of B Lymphocytes for Cell Therapy

In various embodiments, improved methods of expanding populations of engineered B Lymphocytes are contemplated. Said methods involve cross-linking of CD40 expressed on B cells through cell media comprising CD40 ligand and cross-linking antibodies. Combining CD40L together with IL-4, which is a known growth factor for activated B cells has been shown to be crucial for effective activation and proliferation of B cells. Banchereau, J. et al. (1991) SCIENCE 251: 70-72; Schultze, J. L. et al. (1997) J. CLIN. INVEST. 100: 2757-2765. Likewise, IL-21 has also been reported to be an effective stimulus of B cell activation and proliferation. However, additionally, IL-21 drives B cell maturation towards a plasma cell phenotype. Notably, the cell media and methods of various embodiments of the present invention enable engineered B cells to achieve an activation and proliferation outcome, which is comparable to the classical, feeder cell-based NIH3T3/tCD40L protocol.

### 1. CD-40 Ligand

In various embodiments, the engineered B cells are expanded in the presence of CD40 ligand (“CD40L”). In various embodiments, the CD40L is

(SEQ ID NO: 1)
MQKGDQNPQIAAHVISEASSKTTSVLQWAEKGYYTMSNNLVTLENGKQL
TVKRQGLYYIYAQVTFCSNREASSQAPFIASLCLKSPGRFERILLRAAN
THSSAKPCGQQSIHLGGVFELQPGASVFVNVTDPSQVSHGTGFTSFGLL
KL

In various embodiments, the engineered B cells are expanded in the presence of a fusion protein comprising a CD40L sequence and a multimerizing domain. In various embodiments, the multimerizing domain is derived from tenanscin. In various embodiments, the multimerization domain comprises:

(SEQ ID NO: 2)
ACGCAAAPDIKDLLSRLEELEGLVSSLREQ

In various embodiments, the mulitmerization domain is SEQ ID NO: 2 and the CD40L is SEQ ID NO: 1. In various embodiments the fusion protein comprising a multimerization domain and a CD40L comprises:

(SEQ ID NO: 3)
VGDGSSHHHHHHSSGGGRGSHHHHHHGGACGCAAAPDIKDLLSRLEELE
GLVSSLREQGGGSGGGSGGGSMQKGDQNPQIAAHVISEASSKTTSVLQW
AEKGYYTMSNNLVTLENGKQLTVKRQGLYYIYAQVTFCSNREASSQAPF
IASLCLKSPGRFERILLRAANTHSSAKPCGQQSIHLGGVFELQPGASVF
VNVTDPSQVSHGTGFTSFGLLKL.

In preferred embodiments, the CD40L fusion protein comprises an amino acid sequence at least 85% identical to SEQ ID NO. 3. In various embodiments, the CD40L fusion protein comprises an amino acid sequence at least 95% identical to SEQ ID NO. 3. In various embodiments, the CD40L comprises an amino acid sequence of SEQ ID NO. 3.

### 2. Crosslinking Agent

It is contemplated that the B cells of the present invention will be expanded in the presence of a CD40L crosslinking agent. In various embodiments, the CD40L crosslinking agent is an antibody. In various embodiments, the light chain region of the crosslinking antibody comprises:

(SEQ ID NO: 4)
DIVMTQSPSSLSVSAGEKVTMNCKSSQSLLNSGNQRNYLAWYQQKPGQP
PKLLIHGASTRESGVPDRFTGSGSGTDFTLTISSVQAEDLAVYYCQNDH
RYPLTFGAGTKLELKRADAAPTVSIFPPSSEQLTSGGASVVCFLNNFYP
KDINVKWKIDGSERQNGVLNSWTDQDSKDSTYSMSSTLTLTKDEYERHN
SYTCEATHKTSTSPIVKSFNRNEC

In various embodiments, the light chain variable region of the crosslinking antibody comprises:

(SEQ ID NO: 5)
DIVMTQSPSSLSVSAGEKVTMNCKSSQSLLNSGNQRNYLAWYQQKPGQP
PKLLIHGASTRESGVPDRFTGSGSGTDFTLTISSVQAEDLAVYYCQNDH
RYPLTFGAGTKLELK

In various embodiments, the heavy chain region of the crosslinking antibody comprises:

(SEQ ID NO: 6)
EVQLQQFGAELVKPGASVKISCKASGYTFTDYNMDWVKQSHGKSLEWIG
DINPNYGSTSYNQKFKGKATLTVDKSSSTAYMELRSLTSEDTAVYYCAR
DWTGAMDYWGQGTSVTVSSAKTTPPSVYPLAPGCGDTTGSSVTLGCLVK
GYFPESVTVTWNSGSLSSSVHTFPALLQSGLYTMSSSVTVPSSTWPSQT
VTCSVAHPASSTTVDKKLEPSGPISTINPCPPCKECHKCPAPNLEGGPS
VFIFPPNIKDVLMISLTPKVTCVVVDVSEDDPDVQISWFVNNVEVHTAQ
TQTHREDYNSTIRVVSTLPIQHQDWMSGKEFKCKVNNKDLPSPIERTIS
KIKGLVRAPQVYILPPPAEQLSRKDVSLTCLVVGFNPGDISVEWTSNGH
TEENYKDTAPVLDSDGSYFIYSKLNMKTSKWEKTDSFSCNVRHEGLKNY
YLKKTISRSPGK

In various embodiments, the light chain variable region of the crosslinking antibody comprises:

(SEQ ID NO. 7)
EVQLQQFGAELVKPGASVKISCKASGYTFTDYNMDWVKQSHGKSLEWIG
DINPNYGSTSYNQKFKGKATLTVDKSSSTAYMELRSLTSEDTAVYYCAR
DWTGAMDYWGQGTSVTVSS

In various embodiments, the antibody comprises a light chain variable region comprising the amino acid sequence at least 95% identical to SEQ ID NO. 5 and a heavy chain variable region comprising the amino acid sequence at least 95% identical to SEQ ID NO. 7. In various embodiments, the antibody comprises a light chain variable region comprising the amino acid sequence of SEQ ID NO. 5 and a heavy chain variable region comprising the amino acid sequence of SEQ ID NO. 7. In various embodiments, the antibody comprises the light chain of SEQ ID NO. 4 and the heavy chain of SEQ ID NO. 6. In various embodiments, the antibody comprises a light chain comprising the amino acid sequence at least 85% identical to SEQ ID NO. 4 and the heavy chain comprising the amino acid sequence at least 85% identical to SEQ ID NO. 6. In various embodiments, the antibody comprises a light chain comprising the amino acid sequence at least 95% identical to SEQ ID NO. 4 and the heavy chain comprising the amino acid sequence at least 95% identical to SEQ ID NO. 6.

### IV. Engineered B Cells

As used herein, the term “engineered B Cell” refers to a B cell that has been genetically altered to express a desired protein or molecule. Such protein or molecule may be an endogenous or chimeric receptor. Such Engineered B cells may be genetically altered to express a “homing” receptor, which targets a specific tissue/organ type or targets a tumor or specific cell type.

### 1. Antigens

Tumor Antigens. In certain embodiments, the site/target of interest for the B cell is a tumor antigen. The selection of the antigen-binding domain (moiety) of the invention will depend on the particular type of cancer to be treated. Some tumor antigens may be membrane bound, whereas other may be secreted. For example, a tumor antigen may be secreted and accumulate in the extracellular matrix, or the tumor antigen may be expressed as part of the MHC complex. Tumor antigens are well known in the art and may include, for example, CD19, KRAS, HGF, CLL, a glioma-associated antigen, carcinoembryonic antigen (CEA); (3-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, protein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, mesothelin, EGFR, BCMA, KIT and IL-13.

Infectious Disease Antigens. In certain embodiments, the site/target of interest is an infectious disease antigen against which an immune response may be desired. Infectious disease antigens are well known in the art and may include, but are not limited to, viruses, bacteria, protists, and parasitic antigens, such as parasites, fungi, yeasts, mycoplasma, viral proteins, bacterial proteins and carbohydrates, and fungal proteins and carbohydrates. In addition, the type of infectious disease of the infectious disease antigen is not particularly limited, and may include, but are not limited to, intractable diseases among viral infectious diseases such as AIDS, hepatitis B, Epstein Barr Virus (EBV) infection, HPV infection, HCV infection, etc. Parasitic antigens may include, but are not limited to, the malaria parasite sporozoide protein.

In certain embodiments, the modified B cells express an engineered B cell receptor (CAR-B) comprising an extracellular domain, a transmembrane domain and an intracellular domain. In certain embodiments, the extracellular domain comprises a binding domain and a hinge domain.

In certain embodiments, the extracellular domain comprises a binding domain, such as an scFv, ligand, antibody, receptor, or fragment thereof which allows the modified B cell to target specific target cells by binding to proteins expressed on the surface of those cells. In certain embodiments, the modified tumor cells target and bind to proteins/antigens expressed on the surface of tumor cells. In certain embodiments, the modified B cell further expresses a payload. In certain embodiments, the payload is capable of increasing the number of cross-presenting dendritic cells (DC) in tumors. In certain embodiments, the payload is capable of activating and attracting T cells into tumors. In certain embodiments, the payload is capable of fomenting the formation of tertiary lymphoid structures (TLS) in tumors. In certain embodiments of the invention, the modified B cell expresses both a CAR-B and a payload. In certain embodiments, the CAR-B comprises stimulatory domains that activate expression of the payload when bound to an antigen or protein expressed on the surface of a tumor cell.

### 2. Design and Domain Orientation of Chimeric Antigen Receptors in B Cells (CAR-Bs)

In various embodiments, the invention provides a chimeric B Cell Receptor (CAR-B). It will be appreciated that chimeric B cell receptors (CAR-Bs) are genetically engineered receptors. These engineered receptors can be readily inserted into and expressed by B cells in accordance with techniques known in the art. With a CAR-B, a single receptor can be programmed to both recognize a specific protein or antigen expressed on a tumor cell, and when bound to said protein or antigen elicit an anti-tumor response. In various embodiments, the CAR-Bs serve in part as a homing mechanism to deliver B cells to target tissue.

It will be appreciated that relative to the cell bearing the receptor, the chimeric B cell receptor of the invention will comprise an extracellular domain (which will comprise an antigen-binding domain and may comprise an extracellular signaling domain and/or a hinge domain), a transmembrane domain, and an intracellular domain. The intracellular domain comprises at least an activating domain, preferably comprised of CD79a (Immunoglobulin α), CD79b (Immunoglobulin β), CD40, CD19, CD137, Fcγr2a and/or MyD88. It will further be appreciated that the antigen-binding domain is engineered such that it is located in the extracellular protion of the molecule/construct, such that it is capable of recognizing and binding to its target or targets.

Structurally, it will be appreciated that these domains correspond to locations relative to the immune cell. Exemplary CAR-B constructs in accordance with the invention are set forth in Table 2:

TABLE 2

| Construct | Extracellular | | | | |
|---|---|---|---|---|---|
| Name | Domain | Hinge | TM | Signal 1 | Signal 2 |
| pWF-82 | anti-PSMA | CD8 | CD28 | hCD19 | |
| pWF-83 | anti-PSMA | CD8 | CD28 | hCD40 | |
| pWF-84 | anti-PSMA | CD8 | CD28 | hCD40 | CD79b |
| pWF-85 | anti-PSMA | CD8 | CD28 | hCD40 | CD137 |
| pWF-86 | anti-PSMA | CD8 | CD28 | hCD40 | Fcγr2a |
| pWF-87 | anti-PSMA | CD8 | CD28 | hMyd88 | hCD40 |
| pWF-88 | anti-PSMA | CD8 | CD28 | CD79a | |
| pWF-89 | anti-PSMA | CD8 | CD28 | CD79b | |
| pWF-391 | anti-PSMA | 3x strep II tag | CD28 | CD79b | |
| pWF-394 | anti-Sarcoglycan | 3x strep II tag | CD28 | CD79b | |
| pWF-396 | anti-GPC-3 | CD8 | CD28 | CD79a | |
| pWF-397 | anti-GPC-3 | CD8 | CD28 | CD79b | |
| pWF-460 | anti-GPC-3 | Human IgG1 Fc | CD28 | CD79a | |
| pWF428 | anti-GPC-3 | Human Lambda | Human Lambda | | |
| | | Constant region | Constant region | | |
| pWF429 | anti-GPC-3 | Human IgG1 Fc | Human IgG1 Fc | | |
| pWF-521 | Anti-GPC3 vL- | Human IgG1 Fc | Human IgG1 | Endogenous | |
| | hclambda constant | | | BCR complex | |
| | region-linker-vH- | | | | |
| | hcH1-cH2-cH3 | | | | |
| pWF-533 | Anti-GPC3-vL- | | Human IgG1 | Endogenous | |
| | hcH1 | | (complex with | BCR complex | |
| | | | pWF534) | | |
| pWF-534 | Anti-GPC3-vH- | Human IgG1 Fc | Human IgG1 | Endogenous | |
| | hcKappa-hcH2- | | | BCR complex | |
| | cH3 | | | | |

In various embodiments, chimeric B cell receptors are comprised of an extracellular domain, a transmembrane domain and a cytoplasmic domain. In various embodiments, the cytoplasmic domain comprises an activating domain. In various embodiments, the cytoplasmic domain may also comprise a co-stimulatory domain. In various embodiments, the extracellular domain comprises an antigen-binding domain. In various embodiments, the extracellular domain further comprises a hinge region between the antigen-binding domain and the transmembrane domain.

Extracellular Domain. A number of extracellular domains may be used with the present invention. In various embodiments, the extracellular domain comprises an antigen-binding domain. In various embodiments, the extracellular domain may also comprise a hinge region and/or a signaling domain. In various embodiments, the extracellular domains containing IgG1 constant domain may also comprise either IgG1 (hole) or IgG1 (knob) to facilitate directed cBCR formation.

Antigen-Binding Domain and Binding Domain. As used herein, an “antigen binding domain,” “antigen-binding domain” or “binding domain” refers to a portion of the B-CAR capable of binding an antigen or protein expressed on the surface of a cell. In some embodiments, the antigen-binding domain binds to an antigen or protein on a cell involved in a hyperproliferative disease. In preferred embodiments, the antigen-binding domain binds to an antigen or protein expressed on the surface of a tumor cell. The antigen-binding molecules will be further understood in view of the definitions and descriptions below.

An antigen-binding domain is said to “specifically bind” its target antigen or protein when the dissociation constant (K_{d}) is 1×10⁻⁷ M. The antigen-binding domain specifically binds antigen with “high affinity” when the K_{d} is 1-5×10⁻⁹ M, and with “very high affinity” when the K_{d} is 1-5×10⁻¹⁰ M. In one embodiment, the antigen-binding domain has a K_{d} of 10⁻⁹ M. In one embodiment, the off-rate is <1×10⁻⁵. In other embodiments, the antigen-binding domain will bind to antigen or protein with a K_{d} of between about 10⁻⁷ M and 10⁻¹³ M, and in yet another embodiment, the antigen-binding domain will bind with a K_{d} 1.0-5.0×¹⁰.

An antigen-binding domain is said to be “selective” when it binds to one target more tightly than it binds to a second target.

The term “neutralizing” refers to an antigen-binding domain that binds to a ligand and prevents or reduces the biological effect of that ligand. This can be done, for example, by directly blocking a binding site on the ligand or by binding to the ligand and altering the ligand' s ability to bind through indirect means (such as structural or energetic alterations in the ligand). In some embodiments, the term can also denote an antigen-binding domain that prevents the protein to which it is bound from performing a biological function.

The term “target” or “antigen” refers to a molecule or a portion of a molecule capable of being bound by an antigen-binding molecule. In certain embodiments, a target can have one or more epitopes.

The term “antibody” refers to what are known as immunoglobulins, Y-shaped proteins that are produced by the immune system to recognize a particular antigen. The term “antibody fragment” refers to antigen-binding fragments and Fc fragments of antibodies. Types of antigen-binding fragments include: F(ab′)2, Fab, Fab′ and Fv molecules. Fc fragments are generated entirely from the heavy chain constant region of an immunoglobulin.

Extracellular Signaling Domains The extracellular domain is beneficial for signaling and for an efficient response of lymphocytes to an antigen. Extracellular domains of particular use in this invention may be derived from (i.e., comprise) CD28, CD28T (See e.g., U.S. Patent Application US2017/0283500A1), OX40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, CD79a (Immunoglobulin α), CD79b (Immunoglobulin β), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD1 1a, LFA-1, ITGAM, CD1 1b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof. The extracellular domain may be derived either from a natural or from a synthetic source.

Hinge Domains. As described herein, extracellular domains often comprise a hinge portion. This is a portion of the extracellular domain proximal to the cell membrane. The extracellular domain may further comprise a spacer region. A variety of hinges can be employed in accordance with the invention, including costimulatory molecules as discussed above, as well as immunoglobulin (Ig) sequences a 3× strep II spacer or other suitable molecules to achieve the desired special distance from the target cell. In some embodiments, the entire extracellular region comprises a hinge region. In some embodiments, the hinge region comprises the extracellular domain of CD28, or CD8 or a portion thereof as described herein.

Transmembrane Domains. The B-CAR can be designed to comprise a transmembrane domain that is fused or otherwise linked to the extracellular domain of the B-CAR. It can similarly be fused to the intracellular domain of the B-CAR. In one embodiment, the transmembrane domain that naturally is associated with one of the domains in a B-CAR is used. In some instances, the transmembrane domain can be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention may be derived from (i.e. comprise) CD28, CD28T, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, CD79a (Immunoglobulin α), CD79b (Immunoglobulin β), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD1 1a, LFA-1, ITGAM, CD1 1b, ITGAX, CD1 1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

Optionally, short linkers may form linkages between any or some of the extracellular, transmembrane, and intracellular domains of the B-CAR.

In certain embodiments, the transmembrane domain in the B-CAR of the invention is the CD28 transmembrane domain. In one embodiment, the transmembrane domain in the B-CAR of the invention is a CD8 transmembrane domain.

Intracellular (Cytoplasmic) Domains. The intracellular (IC, or cytoplasmic) domain of the B-CAR receptors of the invention can provide activation of at least one of the normal effector functions of the immune cell.

It will be appreciated that suitable intracellular molecules, include, but are not limited to CD79a (Immunoglobulin α), CD79b (Immunoglobulin β), CD40, CD19, CD137, Fcγr2a and MyD88. Intraceullar molecules may further include CD28, CD28T, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD1 1a, LFA-1, ITGAM, CD1 1b, ITGAX, CD1 1c, ITGB1, CD29, ITGB2, CD18, LFA-1, ITGB7, NKG2D, TNFR2, TRANCE/ RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof. The cytoplasmic signaling sequences within the cytoplasmic signaling portion of the CAR-B of the invention may be linked to each other in a random or specified order.

The term “co-stimulatory” domain or molecule as used herein refers to a heterogenous group of cell surface molecules that act to amplify or counteract initial activating signals of the cell.

In one preferred embodiment, the cytoplasmic domain is designed to comprise the signaling domain of hCD19. In another embodiment, the cytoplasmic domain is designed to comprise the signaling domain of hCD40. In another embodiment, the cytoplasmic domain is designed to comprise the signaling domain of hCD40 and hCD79b. In another embodiment, the cytoplasmic domain is designed to comprise the signaling domain of hCD40 and hCD137. In another embodiment, the cytoplasmic domain is designed to comprise the signaling domain of hCD40 and hFcγr2a. In another embodiment, the cytoplasmic domain is designed to comprise the signaling domain of hCD40 and hMyd88. In another embodiment, the cytoplasmic domain is designed to comprise the signaling domain of hCD79a. In another embodiment, the cytoplasmic domain is designed to comprise the signaling domain of hCD79b. These embodiments are preferably of human origin but may be derived from other species.

B. Modified B Cells that Express Payloads.

In various embodiments of the present invention, a modified B cell is provided that is capable of expressing a payload. As used herein the term “payload” refers to an amino acid sequence, a nucleic acid sequence encoding a peptide or protein, or an RNA molecule, for use as a therapeutic agent. In certain embodiments, the payload is for delivery to the tumor or tumor microenvironment. In certain embodiments, it is desirable that the B cell deliver to the tumor or tumor microenvironment a payload capable of, for example, increasing the number of cross-presenting dendritic cells (DCs) in tumors. Cross-presenting DCs will allow for improved presentation of tumor antigens. In various embodiments, the payload may be capable of activating and attracting T cells into tumors. Activating more T cells in tumors will complement the cross-presenting DCs to remold the tumor environment to have more potent antitumor immune capabilities. Payloads may also foment the formation of tertiary lymphoid structures (TLS) in tumors. Clinical studies have demonstrated that there is a relationship between B cells, TLS and responses to immune checkpoint blockade.

Nonexclusive examples of payloads of the present invention include: IL-1, IL-7, IL-8, IL-10, IL-12, IL-13, IL-17, IL-18, IL-21, interferon α, interferon β, interferon γ, TSLP, CCL21, FLT3L, XCL1, LIGHT (TNFSF14), OX40L, CD137L, CD40L, ICOSL, anti-CD3 antibody, CD47, TIM4-FC, CXCL13, CCL21, CD80, CD40L, IFNα A2, LIGHT, 4-1BBL, MDGF (C19orf10), FGF10, PDGF, agrin, TNF-α, GM-CSF, an anti-FAP antibody, an anti-TGF-β antibody; a TGF-β trap, decoy, or other inhibitory molecule; an anti-BMP antibody; a BMP trap, decoy or other inhibitory molecule.

Signaling for Payload Expression. In various embodiments of the present invention, the payload is expressed in the modified B cell as a DNA construct under the control of an activated transcriptional pathway. In certain embodiments, the expression of the payload is controlled by the Nuclear Factor of Activated T cell (“NFAT”) pathway. The NFAT pathway is a transcription factor pathway activated during an immune response and is activated by the NFκB. In various embodiments, the modified B cell expresses both a payload and a CAR-B. In various embodiments, where the modified B cell expresses both a payload and a CAR-B, the CAR-B may further encode signaling molecules that induce activation of the NFκB pathway. Such molecules include, but are not limited to: CD79a (Immunoglobulin α), CD79b (Immunoglobulin β), CD40, CD19, CD137, Fcγr2a and MyD88.

In various embodiments, the invention relates to isolated B cells that express at least one payload. In various embodiments, the invention relates to isolated B cells that express more than one payload. In various embodiments, the invention relates to isolated B cells that express 1, 2, 3, 4, 5, 6, 7, 8, 9, 10, 11 or 12 different payloads.

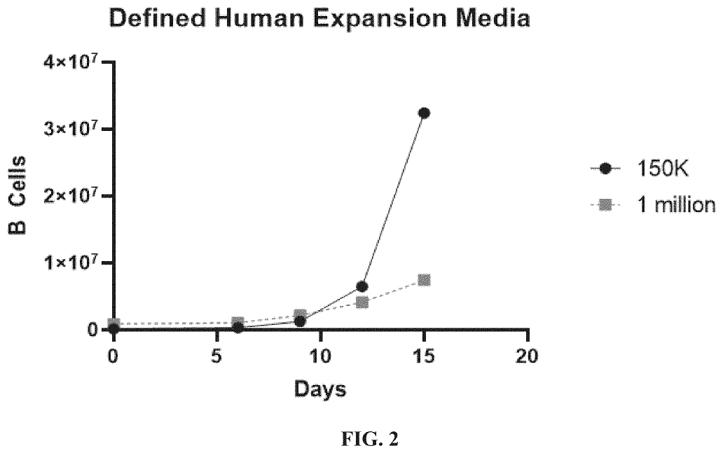

Modification of B Cells for homing. In various embodiments of the present invention, the engineered B cells can be modified with homing domains (e.g., as illustrated in FIG. 2) such that the B cells can home to a site/target of interest and activate upon interaction with the target. Additionally, B cell homing receptors expressed on B cell membranes that recognize addressins and ligands on target tissues, compound or derivatives thereof that alter the trafficking of B cells to a particular site, and inhibitory molecules inflammation and autoimmune activity of the B cells, can play a role in B cell homing and development of specialized immune responses.

Modified B cells that Express Integrin of Interest. The major homing receptors expressed by lymphocytes are the integrins, which are a large class of molecules characterized by a heterodimeric structure of α and β chains. In general, the pairing of specific α and β chains of the integrin determines the type of the homing receptor. For example, pairing of the α4 chain with β7 chain characterizes the major integrin molecule (α4β7) responsible for lymphocyte binding to Mucosal addressin cell adhesion molecule 1 (MAdCAM-1) expressed on high endothelial venules (HEVs) in Peyer's patches (PP) and gastrointestinal (GI) tract lamina propria endothelial venules (LPVs). Similarly, pairing of the α4 chain with β1 chain characterizes the homing receptor (α4β1) for the skin.

In various embodiments of the present inventions, a B cell to be modified can be selected for in advance, with specific traits that mediate preferred localizations. For example, memory B cells expressing CXCR3 may be enriched for and then subjected to engineering. CXCR3 cells may be attracted to ligands expressed at sites of inflammation. As such, modified B cells can preferentially localize to such sites.

In various embodiments of the present invention, a modified B cell is provided that expresses the α4 and β7 chains of an integrin. It is desirable that expression of the α4β7 integrin will promote homing of the modified B cell to the colon. In various embodiments, a modified B cell is provided that expresses the α4 and β1 chains of an integrin. It is desirable that expression of the α4β1 integrin will promote homing of the modified B cell to the skin. In various embodiments, a modified B cell is provided that expresses a desired pairing of an α and a β chain of an integrin, such that the expressed integrin promotes homing of the modified B cell to a desired site/target of interest. Accordingly, in various embodiments, any desired combination of the α and β chains of an integrin is contemplated for expression in the B cells, such that the modified B cells expressing the specific integrin is targeted to a desired site/target of interest.

Modified B cells that Express Homing Receptors of Interest. B cells have an ability to home to inflammatory tissues and altering their homing receptor expression can complement their native homing tendencies. B cell localization is also driven by expression of attractant molecules (e.g., targets such as ligands and chemokines) at inflammatory sites in specific locations or tissues. Such molecules can also include antibodies, such as the MECA79 antibody that targets cells to peripheral node addressin (PNAd). Bahmani et al., J Clin Invest. 2018;128(11):4770-4786; Azzi et al., Cell Rep. 2016;15(6):1202-13. Accordingly, B cells can be engineered to express certain antibodies, proteins, and receptors that facilitate B cell homing to a site/target of interest and interactions of such B cells with the desired target. In certain instances, expression of such receptors redirects the B cells to the tissue of interest.

In various embodiments of the present invention, a modified B cell is provided that is capable of expressing a homing antibody, protein, or a receptor, expression of which is capable of directing the B cell to a specific site/target of interest. Exemplary homing of T cells to specific homing tissues (target tissues) using specific homing receptor/ligand pairs are set forth in Table 3. The same specific homing receptor/ligand pairs are also capable of facilitating homing of B cells to a specific homing tissue (target tissue). Accordingly, in various embodiments of the present invention, homing of the modified B cells to an exemplary homing tissue (target tissue) is facilitated using the corresponding homing receptor/ligand pairs as set forth in Table 3.

TABLE 3
T_{eff} cell homing receptors and their cognate
ligands mediating organotropic targeting

| Homing Tissue Type | T_{eff} Cell Homing Receptor | Cognate Ligand |
|---|---|---|
| Skin | CLA (PSGL-1 glycoform) | E/P-selectin |
| | CD43E | E-selectin |
| | VLA-4 (α₄β₁) | VCAM-1 |
| | LFA-1 (α_{L}β₂) | ICAM-1 |
| | CCR4 | CCL17 |
| | CCR10 | CCL27 |
| Gut (intestine, | α₄β₇ | MAdCAM-1 |
| colon, mLN, PP) | CCR9^{a} | CCL25^{a} |
| | CXCR4 | CXCL12 |
| | Selectin ligands^{b} | E/P-selectin^{b} |
| | VLA-4^{b} | VCAM-1^{b} |
| | LFA-1^{b} | ICAM-1^{b} |
| | CCR6^{b} | CCL20 (MIP-3α)^{b} |
| Liver | CD44 | Hyaluronate |
| | VLA-4 | VCAM-1 |
| | CCR5 | CCL5 |
| | | VAP-1 |
| | Selectin ligands^{b} | E/P-selectin |
| | α₄β₇^{b} | MAdCAM-1^{b} |
| Lung | LFA-1 | ICAM-1 |
| | CCR3 | CCL28 |
| | CCR4 | CCL17 |
| | CXCR4 | CXCL12 |
| | Selectin ligands^{b} | E/P-selectin^{b} |
| | VLA-4^{b} | VCAM-1^{b} |
| | LFA-1^{b} | ICAM-1^{b} |
| Bone Marrow | CLA (PSGL-1 glycoform) | E/P-selectin |
| | CD43E | E-selectin |
| | VLA-4 | VCAM-1 |
| | LFA-1 | ICAM-1 |
| | CXCR4 | CXCL12 |
| | α₄β₇^{b} | MAdCAM-1^{b} |
| Heart | CCR5 | CCL4, CCL5 |
| | CCR4 | ? |
| | CXCR3 | CXCL10 |
| | c-Met | HGF |
| Brain | VLA-4^{b} | VCAM-1^{b} |
| | LFA-1^{b} | ICAM-1^{b} |
| | CXCR3^{b} | CXCL9/CXCL10^{b} |
| Peripheral LN^{c} | Selectin ligands^{b} | E/P-selectin^{b} |
| | LFA-1^{b} | ICAM-1^{b} |
| | CXCR3^{b} | CXCL9/CXCL10^{b} |

^{a}Involved in T_{eff} cell homing to the intestine but not colon.
^{b}Inflammatory reactions, tissue injury.
^{c}Under non-inflamed, steady-state conditions, T_{eff} cells typically lose L-selectin and CCR7 expression and are largely restricted from LN access though may enter during inflammatory reactions (b) as shown. In contrast, both naïve T cells and T_{cm} cells express L-selectin, CCR7, and CXCR4 and engage PNAd, CCL19/CCL21, and CXCL12, respectively, to undergo T-cell rolling and LFA-1/ICAM-1/2- mediated adhesion and transmigration into LNs.

Exemplary homing tissue (target tissue) type and ligand or chemokine that enables tissue-restricted B cell homing in accordance with the invention are set forth in Table 4.

TABLE 4

| Homing Tissue Type | Ligand/Chemokines |
|---|---|
| CNS | VCAM-1, CD62P, ligands for CCR1, |
| | 2, 5, CXCR3 |
| Liver | CD62P, VAP-1, CXCL16 |
| Small Intestine | MAdCAM, CD62P, CCL25 |
| Colon | MAdCAM, CD62P, CCL20, GPR15L |
| Skin | CD62E, CD62P, CCL17(22), ICAM-1 |
| Thymus | VCAM, CD62P, CCL25 |
| Peripheral Lymph Node | PNAd, CCL21, ICAM-1 |
| Peyer's Patch | MAdCAM, CCL21, CXCL13 |
| Bone Marrow | VCAM, CD62P, CXCL12, ICAM-1 |

In various embodiments of the present invention, a modified B cell is provided that expresses one or more of an antibody, a protein, or a receptor that facilitate homing of the modified B cell to the exemplary target/homing tissues using the specific homing receptor/ligand pairs as set forth in Table 3. In various embodiments of the present invention, a modified B cell is provided that expresses one or more of a homing receptor that facilitate homing of the modified B cell to the exemplary target/homing tissue using the ligand or chemokines are set forth in Tables 3 and/or 4. As used herein, the term “B cell homing” refers to localizing, targeting, trafficking, directing, or redirecting of the B cell of the present application to a site/target of interest, for example, a homing or target tissue, an inflammatory site in a specific location or tissue, or a tumor or tumor microenvironment, where delivery of therapeutic payloads is desirable. As used in the context of B cell homing, the term “antibody”, “protein” or a “receptor” refers to an amino acid sequence, a nucleic acid sequence encoding a peptide or protein, or an RNA molecule, for use as a therapeutic agent, which when expressed in a modified B cell of the present invention will direct the B cell to a site/target of interest.

In certain embodiments, the homing antibody, protein, or receptor molecule is for homing/targeting the modified B cell expressing such a molecule to a site/target of interest. In certain embodiments, the homing antibody, protein, or receptor molecule is for homing/targeting the modified B cell expressing such a molecule to inflammatory sites in specific locations or tissues. In certain embodiments, the homing antibody, protein or receptor is for targeting the B cell to a tumor or tumor microenvironment. In certain embodiments, targeting B cells to particular locations is desirable so that the engineered or modified B cells of the present invention can deliver therapeutic payloads to desired locations of interest, for example, a homing or target tissue, an inflammatory site in a specific location or tissue, or a tumor or tumor microenvironment. Accordingly, in certain embodiments, it is desirable that the B cells home to a site/target of interest, for example, a tumor or tumor microenvironment, and deliver to the site/target of interest a payload capable of, for example, increasing the number of cross-presenting dendritic cells (DCs) at the site/target of interest (e.g., in tumors).

In various embodiments, the homing antibody, protein, or receptor is expressed in the modified or engineered B cell as a DNA construct. In various embodiments, the homing antibody, protein, or receptor is expressed in the modified B cell as a DNA construct under the control of a constitutively activated transcriptional pathway. In various embodiments, the homing antibody, protein, or receptor involved in the B cell homing/targeting is either not naturally expressed in a B cell or is expressed at higher levels than is naturally expressed in a B cell. Exemplary homing of the modified B cells to specific homing/target tissues using specific homing receptor/ligand pairs in accordance with the present invention is set forth in Table 4. It should be understood that, notwithstanding the exemplary homing tissues, homing receptor, and ligand pairs set forth in Table 4, a modified B cell of the present invention may be engineered to express any homing antibody, protein, or a receptor (e.g., any homing receptor set for in Table 5), such that the modified B cell can be directed to a specific site/target of interest.

TABLE 5

| Homing Tissue Type | Homing Receptor | Ligand/Chemokine |
|---|---|---|
| Liver | CXCR6 | CXCL16 |
| Small Intestine | CCR9 | CCL25 |
| Large Intestine (Colon) | CCR6 | CCL20 |
| Lymph Node | CCR7 | CCL21 |
| Bone Marrow | CXCR4 | CXCL12 |
| Peyer's Patch | CCR7 and CXCR5 | CCL21 and CXCL13, |
| | | respectively |
| Skin | CCR4 | CCL17(22) |

Nonexclusive examples of homing (target) tissue types for the specific homing receptor/ligand pairs of the present invention include: skin, gut (intestine, colon, mesenteric lymph nodes (mLN), Peyer's Patch (PP), small intestine), liver, lung, bone marrow, heart, peripheral lymph node (LN), CNS, thymus, and bone marrow.

Nonexclusive examples of homing receptors that can be paired with specific or corresponding attractants/ligands/chemokines of the present invention include: CLA (PSGL-1 glycoform), CLA (PSGL-1 glycoform), CCR10, CCR3, CCR4, CCR5, CCR6, CCR9, CD43E, CD44, c-Met, CXCR3, CXCR4, LFA-1, LFA-1 (αLβ2), Selectin ligands, VLA-4, VLA-4 (α4β1), and α4β7.

Nonexclusive examples of ligands/chemokines that can be paired with specific or corresponding homing receptors of the present invention include: CXCL16, CCL17, CCL17(22), CCL20 (MIP-3a), CCL21, CCL25, CCL27, CCL28, CCL4, CCL5, CD62E, CD62P, CXCL10, CXCL12, CXCL13, CXCL16, CXCL9/CXCL10, CXCR3, E/P-selectin, E-selectin, GPR15L, HGF, Hyaluronate, ICAM-1, ligands for CCR1,2, 5, MAdCAM, MAdCAM-1, PNAd, VAP-1, VCAM, and VCAM-1.

In certain embodiments of the present invention, a modified B cell is provided that express or have increased expression of the exemplary B cell homing receptors (e.g., as set forth in Table 3), such that the modified B cell is targeted to the corresponding homing tissue of interest that expresses the corresponding ligand/chemokines (e.g., as set forth in Tables 3 and/or 4). In certain embodiments of the present invention, a modified B cell is provided that co-expresses an integrin with a specific α and β chain pairing and a specific B cell homing receptor (e.g., as set forth in Tables 3 and/or 4), expression of which integrin and/or homing receptor promote or facilitate homing/targeting of the modified B cell to a site/target of interest. In some embodiments, a modified B cell is provided that co-expresses an α4β7 integrin and CCR9. It is desirable that co-expression of α4β7 and CCR9 will promote small intestine homing of the modified B cells of the present invention. In some embodiments, a modified B cell is provided that co-expresses an α4β1 integrin and CCR4. It is desirable that co-expression of α4β1 and CCR4 will promote small intestine homing of the modified B cells of the present invention.

Modified B cells that Express Immune Inhibitory Molecules. B cells are key contributors to many autoimmune diseases. However, B cells can be used therapeutically to antagonize autoimmunity. Specifically, B cells can be engineered to express at least one or more immune inhibitory molecules, which may decrease the autoimmune activity of the B cells, leading to decrease in an autoimmune disease. Immune inhibitory molecules are well known in the art. Such inhibitory molecules may include, but are not limited to, IL-10, TGF-β, PD-L1, PD-L2, LAG-3, and TIM-3. In certain embodiments of the present invention, a modified B cell is provided that is engineered to express at least one or more of an inhibitory molecule selected from IL-10, TGF-β, PD-L1, PD-L2, LAG-3, and TIM-3, or any combinations thereof, such that the inflammation at the site and autoimmune activity of the B cells localized to the site are decreased, thereby leading to a positive therapeutic response.

Compounds that alter B cell Trafficking. In certain embodiments of the present invention, a modified B cell is provided that is treated with at least one or more compound or derivatives thereof that alter the trafficking of B cells by inducing expression of a specific B cell integrin and/or a homing receptor. Compounds or derivatives thereof that alter the trafficking of B cells are well known in the art. In certain embodiments, a modified B cell is provided that is treated with all-trans-retinoic acid (ATRA) or derivatives thereof that promote homing of the B cells to gut (small intestine) due to the increased expression of α4β7 integrin and CCR9 homing receptor. As used herein, the term “compound” refers to a chemical, drug, a therapeutic agent, or derivatives thereof, that alter the trafficking of B cells in a desired manner.

In various embodiments of the present invention, a modified B cell engineered to co-express a specific integrin (e.g., with a specific α and β chain pairing) and a specific B cell homing receptor of interest is treated with at least one or more compounds or derivatives thereof that alter the trafficking of the modified B cells and promote homing of the cells to a specific site/target of interest due to the increased expression of the specific integrin and/or the homing receptor. In various embodiments, a B cell modified to co-express an integrin with a specific α and β chain pairings and a specific B cell homing receptor further expresses at least one or more immune inhibitory molecules, such that the autoimmune activity of the modified B cells targeted to a specific site of inflammation is decreased, leading to a decrease in the autoimmune disease. In some embodiments, a modified B cell engineered to express one or more immune inhibitory molecules, for example IL-10, TGF-β, PD-L1, PD-L2, LAG-3, and TIM-3, or combinations thereof, is treated with ATRA or derivatives thereof for a specified period of time, such that expression of the α4β7 integrin and CCR9 homing receptor is induced to promote B cell homing to a specific site/target of interest (e.g., the gut), but the inflammation at the site and autoimmune activity of B cells localized to the site are decreased, leading to a positive therapeutic response. In one embodiment, a modified B cell engineered to express one or more immune inhibitory molecules, for example IL-10, TGF-β, or combinations thereof, is treated with ATRA or derivatives thereof for a specified period of time, such that expression of the α4β7 integrin and CCR9 homing receptor is induced to promote B cell homing to a specific site/target of interest (e.g., the gut), but the inflammation at the site and autoimmune activity of B cells localized to the site are decreased, leading to a positive therapeutic response.

It is understood that, any B cell of the present invention modified to co-express a specific B cell integrin and homing receptor that targets the B cell to a particular homing/target tissue of interest, may be further engineered to express one or more immune inhibitory molecules for reducing inflammation and autoimmune activity of the B cells localized to the site, and/or treated with a compound that alter the homing/targeting of the modified B cells by inducing expression of the specific B cell integrin and/or the homing receptor.

Activation of B cells with TLR agonists and TLRs. B cells have a natural ability to uptake and present antigens recognized by their specific B cell receptors (BCRs). B cells activated by Toll-like receptors (TLRs) result in potent effector B cells in defending the body in an immune response. Expression of or increasing the expression of TLRs in B cells can provide a mechanism for potentiating B cells for innate signals regulating adaptive immune responses.

Activation of B cells with TLR agonists. In various embodiments of the present invention, a B cell is provided, where the B cell is treated in vitro or ex vivo with at least one TLR agonist. In various embodiments, the TLR can be a TLR1, TLR2, TLR3, TLR4, TLR5, TLR6, TLR7, TLR8, TLR9, TLR10, TLR11, TLR12, and/or a TLR13. In various embodiments, the TLR agonist preferentially binds to one or more TLR selected from the group consisting of TLR1, TLR2, TLR3, TLR4, TLR5, TLR6, TLR7, TLR8, TLR9, TLR10, TLR11, TLR12, and TLR13. TLR agonists are well known in the art and may include, but are not limited to, CpG-rich oligonucleotides and the double-stranded RNA mimic, polyinosinic acid:polycytidylic acid (poly-I:C). In various embodiments, the TLR agonist can be CpG oligonucleotides.

In various embodiments, each B cell may be treated with one TLR agonist. In various embodiments, each B cell may be treated with more than one TLR agonist. For example, each B cell may be treated 1, 2, 3, 4, 5, 6, 7, 8, 9, 10, 11, or 12 different TLR agonists. Alternatively, the patient may be administered a heterogeneous population of B cells, each B cell treated with a unique TLR agonist or a combination of TLR agonists. In some embodiments, the B cells for use a therapeutic agent is treated with one or more TLR agonists at the same time or in advance of the administration of the B cells to a subject or patient in need thereof. In certain embodiments, treatment with one or more TLR agonist is capable of producing more potent effector B cells for defending the body in an immune response. In certain embodiments, treatment with one or more TLR agonist is capable of potentiating B cells for immune responses. In some embodiments, treating a B cell of the present invention with at least one or more TLR agonists induces expression or activation of one or more TLRs.

Activation of B cells with TLR Expression. In various embodiments of the present invention, a modified B cell is provided that is capable of expressing a constitutively active TLR. In various embodiments, the TLR is expressed in the modified or engineered B cell as a DNA construct under the control of a constitutively activated transcriptional pathway. In various embodiments, the TLR is either not naturally expressed in a B cell or is expressed at higher levels than is naturally expressed in a B cell. In various embodiments, the TLR can be a TLR1, TLR2, TLR3, TLR4, TLRS, TLR6, TLR7, TLR8, TLR9, TLR10, TLR11, TLR12, and/or a TLR13.

In various embodiments, each B cell may express more than one constitutively active TLR. For example, each B cell may express 1, 2, 3, 4, 5, 6, 7, 8, 9, 10, 11, 12 or 13 different constitutively active TLRs. Alternatively, the patient may be administered a heterogeneous population of B cells, each B cell capable of expressing and/or secreting a unique TLR or combination of TLRs, which are constitutively active. In various embodiments, 1, 2, 3, 4, 5, 6, 7, 8, 9, 10, 11, 12 or 13 different constitutively active TLRs may be administered to the subject or patient through a heterogeneous population of B cells.

In certain embodiments of the present invention, the B cell is a modified B cell that expresses at least one constitutively active TLR. In certain embodiments, the modified B cell that expresses at least one constitutively active TLR is treated with one or more TLR agonist. In certain embodiments, the expression of the constitutively active TLR is capable of producing more potent effector B cells for defending the body in an immune response. In certain embodiments, the expression of the constitutively active TLR is capable of potentiating B cells for immune responses. In certain embodiments, the modified B cell expresses both a TLR that is constitutively active and any CAR-B of the present application. In various embodiments, the modified B cell expressing a TLR that is constitutively active and/or a CAR-B is further treated with one or more TLR agonist at the same time or in advance of the administration of the modified B cells to a subject or patient in need thereof. In certain embodiments, B cells may be engineered to express payloads and modifiers, such as TLRs, in the absence of CAR-B, for intratumoral administration.

Modified B Cells that Present Antigens Simultaneously in HLA Class I and Class II Molecules. B cells, in addition to their function in antibody production, also express high level of Human Leukocyte Antigen (HLA) class II molecules and can present antigens to CD4+T cells. Hong et al., 2018, Immunity 49, 695-708. In various embodiments of the present invention, a modified B cell is provided that is capable of presenting specific antigens and/or antigen-derived epitopes of interest, such as tumor antigens or infectious disease antigens, simultaneously in both HLA class I and class II molecules. Tumor antigens and infectious disease antigens are well known in the art and are described in the foregoing sections. In certain embodiments, a specific antigen of interest, e.g., a tumor antigen or an infectious disease antigen, is fused to a targeting signal of a lysosomal protein that targets the antigen to the lysosomes and presents the antigen simultaneously and efficiently in both HLA class I and class II molecules. In some embodiments, the targeting signal is the targeting signal of lysosome-associated membrane protein-1 (LAMP1). In some embodiments, the targeting signal is capable entering endosomal recycling compartments. The c-terminal sequence of Clec9A is such a targeting moiety. As used herein, a specific tumor antigen or an infectious disease antigen fused to a targeting signal refers to an amino acid sequence, a nucleic acid sequence encoding a peptide or protein, or an RNA molecule (e.g., an mRNA molecule), for use as a therapeutic agent. In one embodiment, a specific tumor antigen or an infectious disease antigen fused to a targeting signal refers to an mRNA molecule for use as a therapeutic agent. In certain embodiments, it is desirable that the specific tumor antigens and/or infectious disease antigens fused to a targeting signal, such as the targeting signal of LAMP1 or Clec9A, be targeted to the lysosomes or endosomes and presented simultaneously and efficiently in both HLA class I and class II molecules. In certain embodiments, it is desirable that electroporation of B cells (e.g., human B cells), before or after maturation, with an mRNA encoding specific tumor antigens and/or infectious disease antigens of interest fused to a targeting signal, such as the targeting signal of LAMP1 or Clec9A, be capable of simultaneously and efficiently presenting the specific antigens and/or antigen-derived epitopes in both HLA class I and class II molecules. In various embodiments, the specific tumor antigens and/or infectious disease antigens of interest is either not naturally presented by a B cell, is not presented by a B cell simultaneously in both HLA class I and class II molecules naturally, or is not presented by a B cell with high efficiencies in both HLA class I and class II molecules naturally. It is contemplated that, introduction of such electroporated B cells into a subject, e.g., a human host, will promote development of or potentiate antigen-specific immune responses by presenting specific antigens and/or antigen-derived epitopes of interest simultaneously and efficiently in both HLA class I and class II molecules.

In various embodiments, the invention relates to a nucleic acid sequence, e.g., an mRNA sequence, encoding at least one specific antigen of interest, e.g., a tumor antigen or an infectious disease antigen, fused to a targeting signal, such as the targeting signal of LAMP1, for use as a therapeutic agent in electroporation of B cells for simultaneously and efficiently presenting the specific antigen and/or antigen-derived epitopes in both HLA class I and class II molecules. In various embodiments, the invention relates to nucleic acid sequence, e.g., an mRNA sequence, encoding more than one (e.g., 1, 2, 3, 4, 5, or more) specific tumor antigen and/or an infectious disease antigen of interest fused to a targeting signal. In various embodiments, the invention relates to pools of different nucleic acid sequences, e.g., pools of different mRNA sequences, for use as a therapeutic agent in electroporation of B cells as described above, where each pool encodes at least one specific antigen of interest, e.g., a tumor antigen or an infectious disease antigen, fused to a targeting signal that is different from the other pools of the mRNA sequences. Accordingly, in some embodiments, the subject may be administered a homogeneous population of B cells, where each B cell is electroporated with an mRNA encoding at least one specific antigen of interest fused to a targeting signal. In some embodiments, the subject may be administered a homogeneous a population of B cells, where each B cell is electroporated with an mRNA encoding more than one specific antigen of interest fused to targeting signal. In some embodiments, the subject may be administered a heterogeneous population of B cells, where each B cell is electroporated with a combination of mRNAs each encoding at least one specific antigen of interest fused to a different targeting signal.

In some embodiments, the B cells for use in electroporation as described above me be any of the modified B cells of the present application. In some embodiments, the modified B cell comprises a chimeric antigen receptor for B cells (CAR-B). In various embodiments, the modified B cell can express a CAR-B and simultaneously and efficiently present specific antigen and/or antigen-derived epitopes of interest in both HLA class I and class II molecules.

In various embodiments, the invention relates to a method of administering an isolated B cell to a patient in need thereof. In various embodiments, a population of B cells may be administered to the patient. In various embodiments, each B cell may express more than one payload peptide or protein. For example, each B cell may express 1, 2, 3, 4, 5, 6, 7, 8, 9, 10, 11 or 12 different payloads. Alternatively, the patient may be administered a heterogeneous population of B cells, each B cell capable of expressing and/or secreting a unique payload or combination of payloads. In various embodiments, 1, 2, 3, 4, 5, 6, 7, 8, 9, 10, 11 or 12 different payloads may be administered to the patient through a heterogeneous population of B cells.

### V. Methods of Treatment

In various aspects of the invention, the expanded population of B cells will be delivered as a therapeutic to a patient in need thereof. In various embodiments, the expanded population of B cells will be capable of treating or preventing various diseases or disorders, including cancer.

In some embodiments, the invention relates to creating a B cell-mediated immune response in a subject, comprising administering an effective amount of the expanded and/or engineered B cells of the present application to the subject. In some embodiments, the B cell-mediated immune response is directed against a target cell or cells. In some embodiments, the engineered immune cell comprises a chimeric antigen receptor for B cells (B-CAR). In some embodiments, the target cell is a tumor cell. In some aspects, the invention comprises a method for treating or preventing a malignancy, said method comprising administering to a subject in need thereof an effective amount of at least one isolated antigen-binding molecule described herein. In some aspects, the invention comprises a method for treating or preventing a malignancy, said method comprising administering to a subject in need thereof an effective amount of at least one immune cell, wherein the immune cell comprises at least one chimeric antigen receptor.

In some aspects, the invention comprises a pharmaceutical composition comprising an expanded population of engineered B cells comprising at least one antigen-binding molecule as described herein and a pharmaceutically acceptable excipient. In some embodiments, the pharmaceutical composition further comprises an additional active agent.

In some embodiments, the subject is diagnosed with a metastatic disease localized to the liver. In other embodiments, the metastatic disease is a cancer. In still other embodiments, the cancer metastasized from a primary tumor in the breast, colon, rectum, esophagus, lung, pancreas and/or stomach. In still other embodiments, the subject is diagnosed with unresectable metastatic liver tumors. In yet other embodiments, the subject is diagnosed with unresectable metastatic liver tumors from primary colorectal cancer. In some embodiments, the subject is diagnosed with hepatocellular carcinoma.

It will be appreciated that target doses for modified B cells can range from 1×10⁶-2×10¹⁰ cells/kg, preferably 2×10⁶ cells/kg, more preferably. It will be appreciated that doses above and below this range may be appropriate for certain subjects, and appropriate dose levels can be determined by the healthcare provider as needed. Additionally, multiple doses of cells can be provided in accordance with the invention.

Also provided are methods for reducing the size of a tumor in a subject, comprising administering to the subject a modified B cell of the present invention, wherein the cell comprises a CAR-B receptor comprising an antigen-binding domain that binds to an antigen on a tumor, a payload or both a CAR-B and a payload. In some embodiments, the subject has a solid tumor, or a blood malignancy such as lymphoma or leukemia. In some embodiments, the modified B cell is delivered to a tumor bed. In some embodiments, the cancer is present in the bone marrow of the subject.

Also provided are methods for homing B cells to a site/target of interest in a subject, comprising administering to the subject a modified B cell of the present invention, wherein the cell comprises an integrin, a homing antibody, protein, or a receptor that is attracted to a ligand, chemokine, or an attractant at the site/target of interest. In some embodiments, the site/target of interest is, for example, a homing or target tissue, an inflammatory site in a specific location or tissue, or a tumor or tumor microenvironment, where delivery of therapeutic payloads is desirable.

Also provided are methods for decreasing inflammation and autoimmune activity of B cells at a site/target of interest in a subject, comprising administering to the subject a modified B cell of the present invention, wherein the cell comprises an immune inhibitory molecule. In some embodiments, the site/target of interest is, for example, a homing or target tissue, an inflammatory site in a specific location or tissue, or a tumor or tumor microenvironment, where delivery of therapeutic payloads is desirable.

In some embodiments, the expanded population of engineered B cells are autologous B cells. In some embodiments, the modified B cells are allogeneic B cells. In some embodiments, the modified B cells are heterologous B cells. In some embodiments, the modified B cells of the present application are transfected or transduced in vivo. In other embodiments, the engineered cells are transfected or transduced ex vivo.

As used herein, the term “subject” or “patient” means an individual. In some aspect, a subject is a mammal such as a human. In some aspect, a subject can be a non-human primate. Non-human primates include marmosets, monkeys, chimpanzees, gorillas, orangutans, and gibbons, to name a few. The term “subject” also includes domesticated animals, such as cats, dogs, etc., livestock (e.g., llama, horses, cows), wild animals (e.g., deer, elk, moose, etc.,), laboratory animals (e.g., mouse, rabbit, rat, gerbil, guinea pig, etc.) and avian species (e.g., chickens, turkeys, ducks, etc.). Preferably, the subject is a human subject. More preferably, the subject is a human patient.

In certain embodiments, compositions comprising CAR-expressing immune effector cells disclosed herein may be administered in conjunction with any number of chemotherapeutic agents. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, que-lamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as TARGRETIN™ (bexarotene), PANRETIN™, (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (CYTOXAN®) Doxorubicin (hydroxydoxorubicin), Fludarabine, Vincristine (ONCOVIN®), and Prednisone.

A variety of additional therapeutic agents may be used in conjunction with the compositions described herein. For example, potentially useful additional therapeutic agents include PD-1 (or PD-L1) inhibitors such as nivolumab (OPDIVO®), pembrolizumab (KEYTRUDA®), pembrolizumab, pidilizumab, and atezolizumab (TECENTRIQ®).

Additional therapeutic agents suitable for use in combination with the invention include, but are not limited to, ibrutinib (IMBRUVICA®), ofatumumab (ARZERRA®), rituximab (RITUXAN®), bevacizumab (AVASTIN®), trastuzumab (HERCEPTIN®), trastuzumab emtansine (KADCYLA®), imatinib (GLEEVEC®), cetuximab (ERBITUX®), panitumumab (VECTIBIX®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In additional embodiments, the composition comprising CAR-containing immune can be administered with an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®)), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

In certain embodiments, the compositions described herein are administered in conjunction with a cytokine. “Cytokine” as used herein is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1 alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

### EXAMPLES

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### Example 1

### Purification and Enrichment of Human B Cells

Human B cells were collected and enriched from PBMCs according to the following protocol. PBMCs were prepared from buffy coats as follows. Per donor (approx. 25 ml) volume was brought up to 120 ml with PBS. Next, 30 ml was layered onto 15 ml of Ficoll in four 50 ml tubes. This was then spun for 20 minutes at 450 g. The upper layer was then removed and discarded. The PBMC interface, which is just below the first layer, was then isolated (neutrophils are the most dense and increase in number towards bottom of layer). The interface of each of the 4 tubes was taken from each donor and transferred to two 50 ml conical tubes. Each was brought up to 50 ml per tube using PBS. Each of the two tubes was then spun at 500g for 5 minutes.

RBC lysis was performed by aspirating the pellet and bringing up in a 10 ml total volume combined for both pellets of ACK lysis buffer. The solution was then kept for five minutes at room temperature. Next, 40 ml of PBS was added, the solution was mixed, and then centrifuged at 500 g for 5 minutes. This mixture was then brought to 40 ml volume in PBS in order to count the yield of PBMCs. 150 million PBMCs for each B cell enrichment prep were used.

Enrichment of B cells was performed according to instructions for product ID 17954 EASYSEP® purification of human B cells (Stem Cell Technologies). The solution was spun down and 150 million PBMCs were resuspended into 3 ml of EASYSEP® isolation buffer. The pellet was resuspended in 3 ml EASYSEP® buffer in a 15 ml tube. 150 μl of cocktail enhancer was added. 150 μl isolation cocktail was added, and the tubes were mixed and incubated for 5 minutes at room temperature. Rapid spheres were vortexed for 30 sec and 150 μl was added to the tube and then mixed, and moved to the magnet. After 3 minutes, this was poured into a new tube and the magnet step was repeated.

### Example 2

### Expansion of B Cells Using HELA-CD40L Feeder Cells or MEGA-CD40L

Due to the low frequency of B cells in human peripheral blood, it is hard to obtain sufficient numbers of B cells for clinical cell therapy purposes. Therefore, B cell expansion is an important step in manufacturing clinical grade B cells. Human B cells require CD40L for growth. It has been established that B cells can survive and expand if grown on a monolayer of HEL cells expressing CD40L. However, it is necessary to establish methods for B cell growth, which are not dependent on feeder cells. In an effort to determine if commercially available tools ore reagents existed for this purpose, Enzo MEGA-CD40L was tested in comparison with growth on HELA-CD40L feeder cells.

Growth Media Conditions on HeLa-CD40L feeder cells. Feeder cell plates were prepared by irradiating CD40L HeLa cells at 5×10⁶ per plate on a 24 well plate. The base media was comprised of RPMI-1640+10% FCS; Penn/strep (100 u/ml, 100 ug/ml); Sodium Selenite (100nM); and IL-4 (2 ng/ml) (R & D 204-IL). Feeder cells were allowed to grow for at least 24 hours before plating the B cells.

Growth Media Conditions with MEGA-CD40L. The media conditions were the same as above, except that there were no feeder cells. Instead, the B cells were plated in media comprising of RPMI-1640 +10% FCS; Penn/strep (100 u/ml, 100 ug/ml); Sodium Selenite (100nM); IL-4 (2 ng/ml) (R & D 204-IL); and MEGA-CD40L (100 ng/ml).

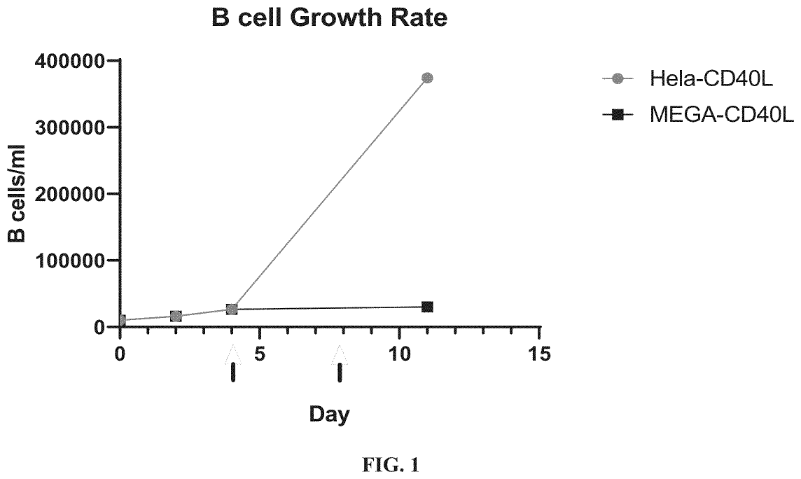

B-cell growth rate in HeLa-CD40L and MEGA-CD40L conditions are depicted in FIG. 1. This study demonstrated that human B cells can be grown and expanded in media with HELA-CD40L feeder cells, but do not expand in the presence of MEGA-CD40L.

The purified B cells were started at equivalent densities on day 0 at approximately 10,000 cells per ml and grown on HELA-CD40L feeder cells. On day 4, the cultures were split into two groups and one group continued to be grown in the same media with HELA-CD40L feeder cells whereas the other group was grown in media supplemented with MEGA-CD40L (0.1 μg/mL) but in the absence of HELA feeder cells. On day 8, an additional 100 ng/mL of MEGA-CD40L was added to the growth media in the MEGA-CD40L condition. Arrows in FIG. 1 indicate the time at which MEGA-CD40L treatment occurred.

The data show that there was significant proliferation of B cells by day 11 when grown on HELA-CD40L feeder cells, but no significant growth observed on day 11 if grown with MEGA-CD40L.

### Example 3

### Expansion of B Cells Without Feeder Cells

In this example, B cells were expanded in the absence of HELA-CD40L expressing feeder cells. B cells were plated in expansion media comprising IL-4 (5 μg; 5000 IU/μg dissolved in 100 μL of H₂O to achieve 2.5×10⁵ IU/ml); CD40L (SEQ ID NO: 140, 500 μg resuspended in 500 μL); a CD40L cross-linking antibody (SEQ ID NOS: 143 and 144); human AB serum (IC092938249 VWR); and 10 μL of penicillin streptomycin. B cells were plated at a density of either (i) 100,000 to 150,000/ml; or (ii) 1,000,000/mL and expanded for at least 15 days, being refed with fresh media every seven days.

Cells maintained at a density of 100,000 to 150,000 cells/mL were able to expand more than 200-fold in two weeks; whereas cells maintained at a density of 1,000,000 cells/ml showed significantly less cell growth. See FIG. 2.

### Example 4

### Engineering of Expanded B Cells

Expanded human B cells were engineered to express various chimeric receptors using an adenovirus vector. B cells were first purified, enriched and expanded using the techniques described in Example 1 and 3. B cells were cultured in the expansion media of Example 3 for 10 days and then transduced with an adenovirus listed in Table 6.

TABLE 6

| | | | Extracellular | | Transmembrane | |
|---|---|---|---|---|---|---|
| Name | Subtype | Promoter | Target | Hinge | Domain | Intracellular |
| IL-10 | Ad5RGD | pMMLV(LTR)- | mIL10 | N/A | | |
| | | hEF1a | | | | |
| cBCR1- | Ad5F35 | | GPC3 | | | |
| GPC3 | | | | | | |
| RGD-478 | Ad5RGD | pMMLV(LTR)- | Anti- | muIgG2a Fc | | |
| | | hEF1a | Sarcoglycan | | | |
| | | | scFv | | | |
| RGD-601 | Ad5RGD | pMMLV(LTR)- | GPC3-scFv | mCD8H | mCD28M | mCD79a |
| | | hEF1a | | | | |
| RGD-463 | Ad5RGD | pMMLV(LTR)- | GPC3-scFv | muIgG2a Fc | | |
| | | hEF1a | | | | |
| RGD-602 | Ad5RGD | pMMLV(LTR)- | GPC3-scFv | muIgG2a FC | | |
| | | hEF1a | | | | |
| RGD-394 | Ad5RGD | pMMLV(LTR)- | GPC3-scFv | mCD8H | mCD28M | mCD79b |
| | | hEF1a | | | | |
| GFP-RGD | Ad5RGD | pMMLV(LTR)- | Anti- | | | |
| | | hEF1a | Sarcoglycan | | | |
| | | | scFv | | | |

| | | |
|---|---|---|
| Empty | Ad5RGD | N/A |
| Vector | | |
| No Virus | N/A | N/A |

The Ad5 adenovirus either comprised The B cells were then cultured in vitro for 10 days using B cell expansion media as described herein. The expanded B cells were then transduced with adenovirus as per the following protocol.

The B cells were grown for 10 days and achieved a yield of approximately 14 million cells. Several Adenovirus F35 virus constructs encoding Human BCR's were tested. One example is as follows. Adenovirus was used encoding a GPC3-specific BCR. Also used was an adenovirus empty vector control. No virus mock control was also used.

20 μl of 10⁹/ml virus particles was added to 0.5 million B cells under the following conditions:

B cells were plated in OPTIMEM® with 0.2% BSA and CD40L+Xlinker (Miltenyi Product #130-098-775, with the same concentration as described in the expansion media outlined above), IL-4 (as described in the expansion media), and 0.5 μg/ml polybrene in 200 μl on a 24-well plate. After adding virus, the plate was spun for 1 hour at 1100 g, then transferred to the incubator for 2.5 hours and given 2 mL of fresh expansion media. After 3 days, the cells were stained with GPC3-BV to detect B cells expressing the BCR. The results are set forth in FIGS. 2A-2C, and show that at least 67% of the cells expressed the GPC3 BCR at 72 hours post transduction.

### Example 5

### Expression of Ad5RGD-GFP in Human B Cells

B cells From PBMC' s were grown in culture for 10 days. The B cells were transduced with Ad5-RGD constructs encoding GFP and placed into OPTIMEM® with 0.2% BSA, CD40L+Xlinker, 5% human AB serum, and IL-4. Virus was then added and spun at 1100 g for 1 hour, then incubated 3 hours at 37° C. Media was then switched to normal human B cell growth media until analysis.

The expression testing results of multiple time points of GFP were as follows:

| | | | | |
|---|---|---|---|---|
| 72 | 120 | 144 | 192 | 240 |

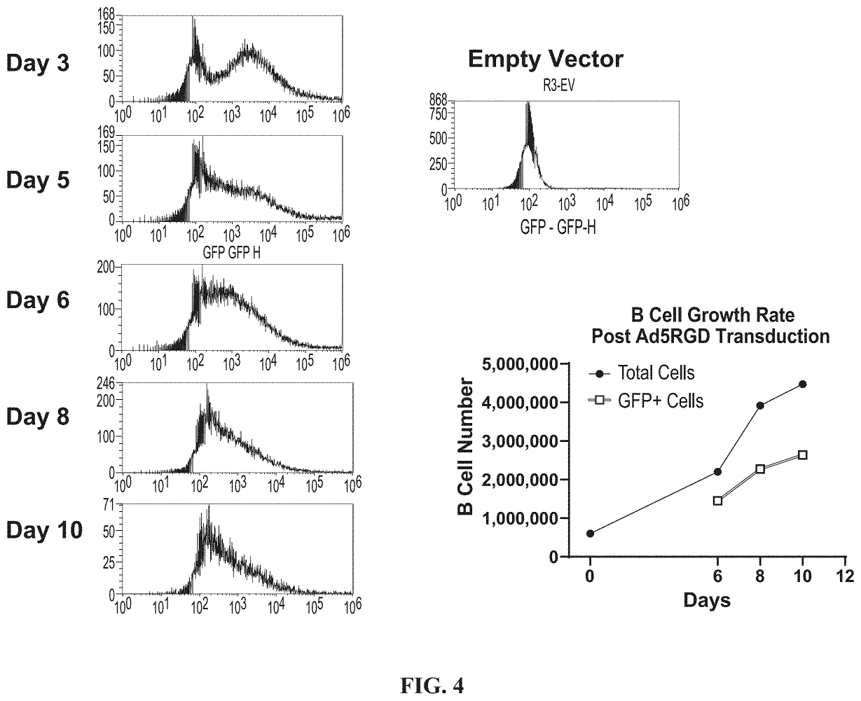

To address impact of virus on cell viability, total cell counts and fraction of GFP positive counts are tested by VICELL® and FACS on day 6, 8, and 10. Results are set forth in FIG. 4. Ad5-RGD modified-GFP transduction results in high efficiency of GFP expression in B cells. Expression was maintained in approximately 60% if total B cells for at least 10 days. Transduced B-cells continued to proliferate for at least one week post-transduction.

### Example 6

### Expression of Murine BCR Constructs or IL-10 in Human B Cells by Transduction with Adenovirus

B cells derived from PBMC's were grown in culture for 10 days. The B cells were then transduced with Ad5-RGD constructs encoding murine IL-10, or several formats of murine BCRs. (Murine was used while waiting on human formats). The B cells were then cultured in conditions comprising OPTIMEM® with 0.2% BSA, CD40L+Xlinker, 5% human AB serum, and IL-4. Virus was added and spun at 1100 g for 1 hour, then incubated 3 hours at 37° C., then switched to normal human B Cell growth media until analyzed. The virus was tested at two ratios (20 μl:0.5 million B cells and 2 μl:0.5 million B cells).

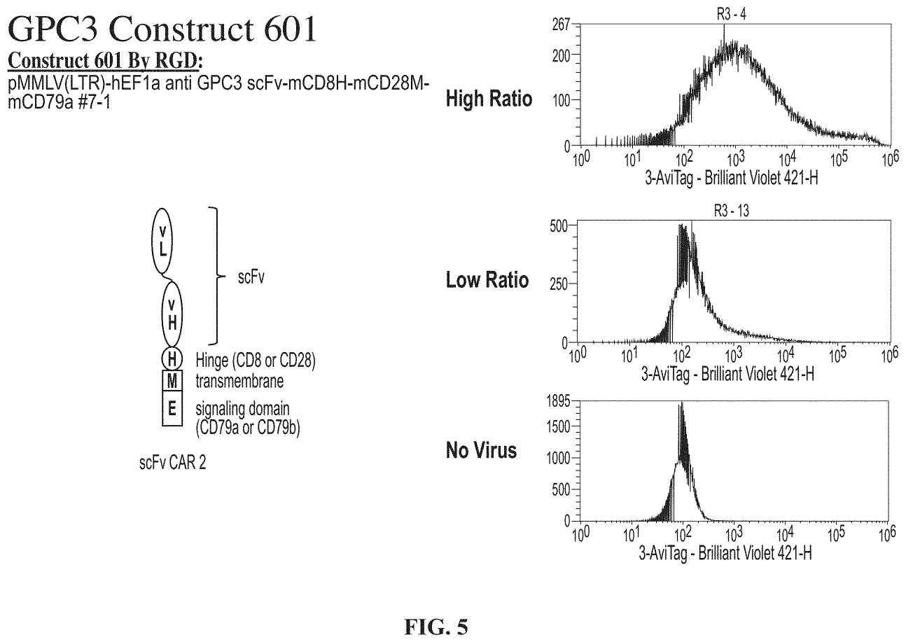

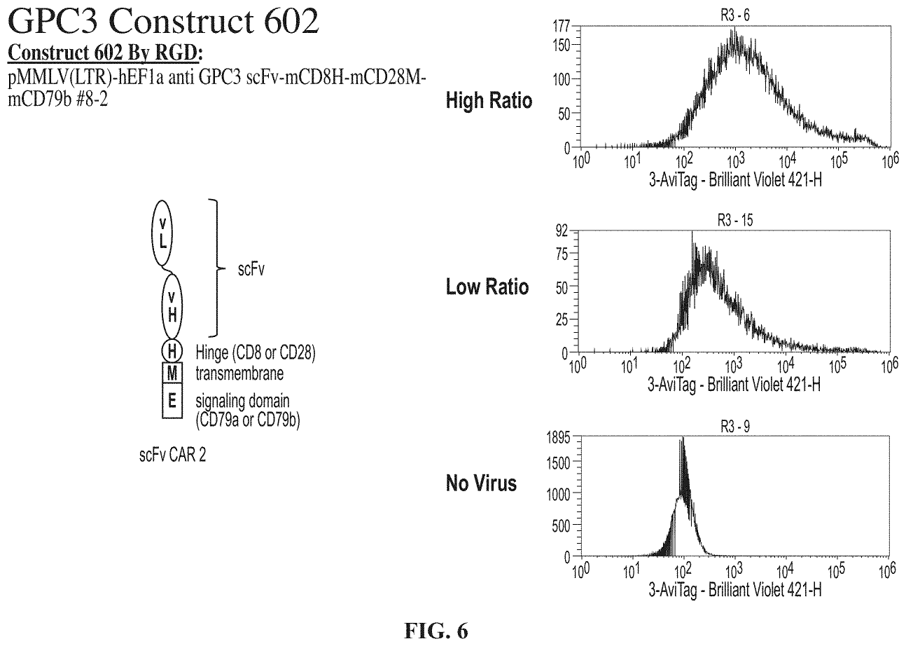

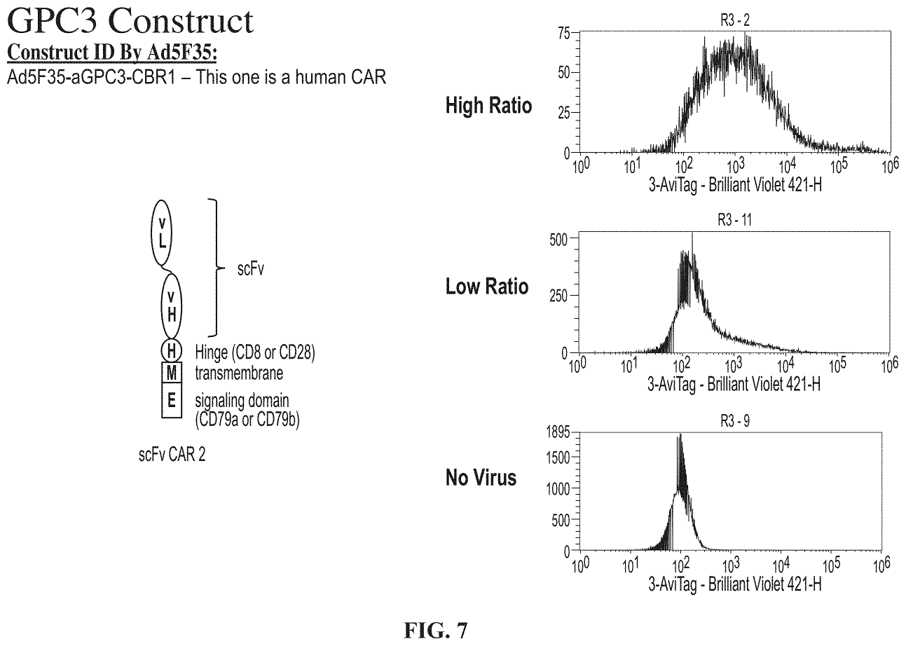

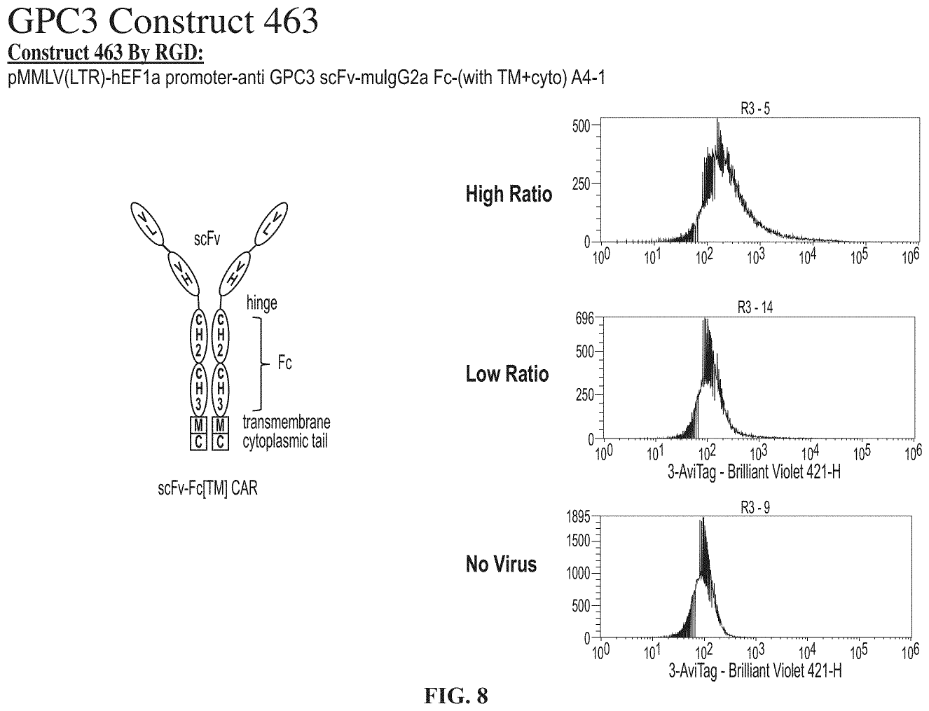

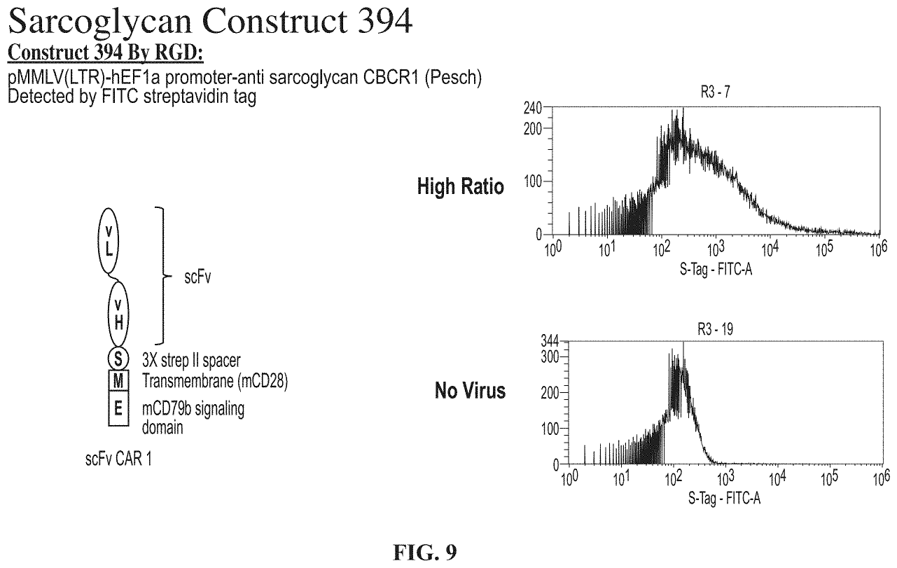

Expression testing was performed on day 4, 96 hours post transduction. IL-10 supernatants were stored at −80° C. GPC3 constructs were detected by binding to human GPC3-BV. Sarcoglycan construct was detected by binding to FITC strep tag. The results are set forth in FIGS. 5 through 9.

### Example 7

### Monitoring In Vivo Homing and Expression Human BCRs in Human B cells After Delivery by Adenovirus

B cells From PBMC's were grown in culture for 10 days. The B cells were then transduced with Ad5f35 constructs encoding luciferase +/−GPC3-BCR. PWF-524/PWWF-684 (Luc+GPC3 CAR) and PWF-684 (Luc) were transduced and then cultured overnight in Wennhold media to allow time for expression of the BCR and Luc. Thereafter, approximately 0.9 million cells were delivered IV to mice with HEPG2 tumors. Luc was monitored by bioluminescence.

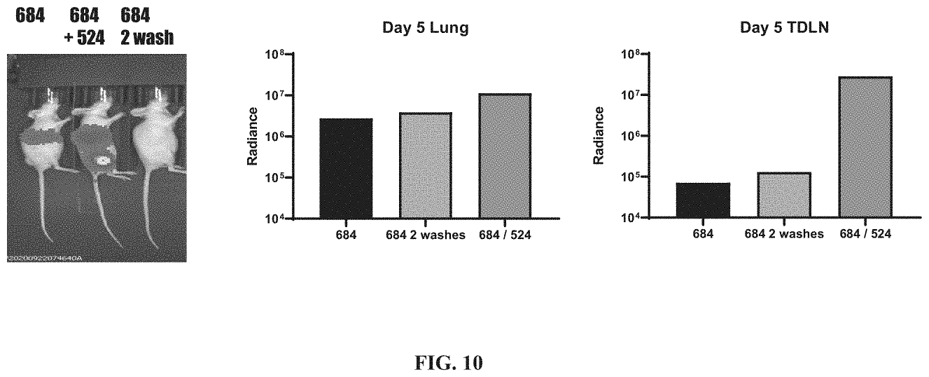

Data are shown in FIG. 10 which shows that GPC3 CAR (524) enriches homing of human B cells 100-Fold to the TDLN of SHORN mice harboring HEPG2 tumors. Specifically, SHORN mice are deficient in T, B, and NK Cells, which allow for human B cells to not be rejected. There was a 100-fold enrichment noted in the TDLN relative to lung. There was a 2-5 fold higher signal observed in the lung, which could possibly have been due to HEPG2 Metastasis. FIG. 10 shows that engineered GPC3 CARS can help instruct B Cells to home to inflamed lymphoid organs.

1. A method of treating a disease or disorder in a subject in need thereof comprising
a. obtaining a population of B cells from a source;
b. culturing said B cells in a culture medium comprising a CD40L fusion protein, and a CD40L cross-linking agent;
c. engineering said B cells to express either a payload, a chimeric receptor or both; and
d. administering said B cells to said subject.

2. The method of claim 1, wherein the source is a mammal.

3. The method of claim 2, wherein said source is a biological sample comprising peripheral mononuclear blood cells.

4. The method of claim 1, wherein the CD40L fusion protein comprises an amino acid sequence at least 85% identical to SEQ ID NO. 3.

5. The method of claim 1, wherein the CD40L fusion protein comprises an amino acid sequence at least 95% identical to SEQ ID No. 3.

6. The method of claim 1, wherein the CD40L fusion protein comprises an amino acid sequence of SEQ ID NO. 3.

7. The method of claim 1, wherein the CD40L crosslinking agent is an antibody.

8. The method of claim 7, wherein the antibody comprises a light chain variable region comprising the amino acid sequence at least 95% identical to SEQ ID NO. 5 and a heavy chain variable region comprising the amino acid sequence at least 95% identical to SEQ ID NO. 7.

9. The method of claim 8, wherein the antibody comprises a light chain variable region comprising the amino acid sequence of SEQ ID NO. 5 and a heavy chain variable region comprising the amino acid sequence of SEQ ID NO. 7.

10. The method of claim 1, wherein the B cell is engineered prior to culturing said B cells in a medium with CD40L fusion protein and a CD40L crosslinking agent.

11. The method of claim 1, wherein the B cell is engineered after culturing said B cells in a medium with CD40L fusion protein and a CD40L crosslinking agent.

12. The method of claim 1, further comprising culturing said B cells in the presence of IL-4.

13. The method of claim 1, further comprising culturing said B cells in the presence of IL-21.

14. The method of claim 1, wherein the cultured B cells express at least one of the following markers: CD62L, CCR7, CD80, CD86, CD54, ICAM, CD58, or CD27.

15. The method of claim 1, wherein the disease or disorder is selected from the group consisting of at least one of cancer, heart disease, inflammatory disease, muscle wasting disease, or neurological disease.

16. The method of claim 15, wherein the cancer is at least one of breast cancer, colon cancer, rectal cancer, esophageal cancer; lung cancer, pancreatic cancer, stomach cancer, liver cancer, hepatocellular carcinoma, stromal tumors such as GIST, glioblastoma, and glioma.

17. The method of claim 1, wherein at least about 3×10⁷ B cells are administered to said subject.

18. The method of claim 1, wherein the population of B cells are cultured for at least 14 days.

19. A method of treating a disease or disorder in a subject in need thereof comprising
a. obtaining a population of B cells from a source;
b. culturing said B cells in a culture medium comprising a CD40L fusion protein, wherein said CD40L fusion protein comprises an amino acid sequence at least 95% identical to the amino acid sequence of SEQ ID NO. 3, and a CD40L cross-linking antibody whose light chain variable region is at least 95% identical to the amino acid sequence of SEQ ID NO: 5 and whose heavy chain variable region is at least 95% identical to the amino acid sequence of SEQ ID NO: 7; and
c. administering said B cells to said subject.

20. The method of claim 19, wherein the source is a mammal.

21. The method of claim 19, wherein the CD40L fusion protein comprises an amino acid sequence of SEQ ID NO. 3.

22. The method of claim 19, wherein the antibody comprises a light chain variable region comprising the amino acid sequence of SEQ ID NO. 5 and a heavy chain variable region comprising the amino acid sequence of SEQ ID NO. 7.

23. The method of claim 19, further comprising engineering said B cells to express either a payload, a chimeric receptor or both.

24. The method of claim 19, wherein the B cell is engineered prior to culturing said B cells in a medium with CD40L fusion protein and a CD40L crosslinking antibody.

25. The method of claim 19, wherein the B cell is engineered after culturing said B cells in a medium with CD40L fusion protein and a CD40L crosslinking antibody.

26. The method of claim 19, further comprising culturing said B cells in the presence of IL-4.

27. The method of claim 19, further comprising culturing said B cells in the presence of IL-21.

28. The method of claim 19, wherein the cultured B cells express at least one of the following markers: CD62L, CCR7, CD80, CD86, CD54, ICAM, CD58, or CD27.

29. The method of claim 19, wherein the disease or disorder is selected from the group consisting of at least one of cancer, heart disease, inflammatory disease, muscle wasting disease, or neurological disease.

30. The method of claim 29, wherein the cancer is at least one of breast cancer, colon cancer, rectal cancer, esophageal cancer; lung cancer, pancreatic cancer, stomach cancer, liver cancer, hepatocellular carcinoma, stromal tumors such as GIST, glioblastoma, and glioma.
